(19) United States
(12) Patent Application Publication
(10) Pub. No.: US 2023/0284912 A1
(43) Pub. Date: Sep. 14, 2023

(54) LONG-TERM CONTINUOUS BIOMETRIC MONITORING USING IN-EAR POD

(71) Applicant: STAT HEALTH INFORMATICS, INC., Waban, MA (US)

(72) Inventors: Daniel LEE, Brighton, MA (US); Paul JIN, Waban, MA (US); Joshua B. MINUSKIN, North Easton, MA (US)

(21) Appl. No.: 18/044,461

(22) PCT Filed: Sep. 10, 2021
(86) PCT No.: PCT/US2021/049841

§ 371 Date: Mar. 8, 2023

Related U.S. Application Data
(60) Provisional application No. 63/077,426, filed on Sep. 11, 2020.

(51) Int. Cl. A61B 5/0205; A61B 5/026; A61B 5/00; A61B 5/11

(52) U.S. Cl. CPC A61B 5/0205; A61B 5/0261; A61B 5/7264; A61B 5/6816; A61B 5/6832; A61B 5/7275; A61B 5/11; A61B 5/02416

(57) ABSTRACT

Provided herein are devices, systems, platforms and methods for long-term, continuous monitoring of a biometric parameter of a subject using an in-ear pod.

### CROSS-REFERENCE TO RELATED APPLICATION

This application claims the benefit of U.S. Provisional Application No. 63/077,426 filed on Sep. 11, 2020, which is hereby incorporated by reference in its entirety.

### BACKGROUND

Poor Cerebral Blood Flow (CBF) is a major public health concern, especially for the elderly. Poor Cerebral Blood Flow most often occurs when a transition to standing causes a reduction of blood flow to the head. Some known diseases, conditions, and syndromes that cause Poor Cerebral Blood Flow include Orthostatic Hypotension (OH), Postural Orthostatic Tachycardia Syndrome (POTS), Orthostatic Cerebral Hypoperfusion Syndrome (OCHOs), Primary Cerebral Autoregulatory Failure (pCAF), Vasovagal Syncope, Carotid Sinus Sensitivity, hypovolemia, drug-induced hypotension, arrhythmias, vascular stenosis, aortic stenosis, Ehlers-Danlos Syndrome, Multiple Sclerosis, Multiple System Atrophy, Parkinson's, dementia, as well as various other neurological disorders that compromise the autonomic system (dysautonomias). Such loss of blood flow often leads to falling, a leading cause of death in the elderly. Approximately 1 in 4 adults over 65 years old fall once in a year causing 4 deaths/hour. Further, 800,000 people are hospitalized each year, and 3 million people are treated in emergency rooms each year, for head injury or hip fracture, requiring an estimated 50 billion dollars in reactive medical costs.

The best method currently available to physicians for diagnosing Cerebral Blood Flow issues is the tilt-table test. This involves having a patient come into the doctor's office to lie supine on a table while heart rate and blood pressure measurements are made. The table is then tilted such that the patient is in an upright position, then more blood pressure measurements are taken. Diagnoses are made based primarily on how the heart rate and blood pressure respond to the tilt and whether the patient experiences presyncope or syncope symptoms. There are many problems with this approach. For one, cardiovascular behavior varies dramatically throughout a single day, as well as throughout the week, yet the tilt table test is a one-time test at the doctor's office whenever the scheduling can work out. In addition, cardiovascular behavior is highly sensitive to what a patient is doing at a given moment, especially sensitive to ‘fight or flight’ sympathetic activation, yet the tilt table has the patient physically strapped down to a rotating table, resulting in test-induced artifacts such as white coat hypertension. Finally, blood pressure is most often measured at the arm with a cuff, whereas blood flow and blood pressure at the head are what actually matter when considering the primary orthostatic symptoms of presyncope and syncope. The shortcomings of the current testing paradigm often mask the presence and/or severity of the patients' condition, and may not accurately reflect what's actually going on at home. Thus, long-term ambulatory monitoring of blood flow and blood pressure at the head is a much better approach to monitor and manage orthostatic issues.

### SUMMARY

Provided herein is an in-ear device for long-term, continuous monitoring of a subject, the device comprising: a biometric sensor configured to monitor at least one biometric parameter of the subject; a movement sensor configured to monitor at least one activity parameter of the subject; a micro energy storage bank; an energy harvesting element configured to charge the micro energy storage bank; a wireless communications transceiver; and an attachment mechanism for securing the device to the auricle of the subject.

In some embodiments, the biometric sensor is located inside the Cymba Concha. In some embodiments, the wearable device comprises one or more biometric sensors, with the wearable device or the one or more biometric sensors located inside the cymba concha of the subject. In some embodiments, the disposition of the wearable device or the one or more biometric sensors within the cymba concha allows for superior signal quality with minimal noise artifacts in part due to strong vascularization coming off branches of the posterior auricular artery, as well as minimal musculature that could introduce noise artifacts. In some embodiments, disposition of the wearable device or the one or more biometric sensors within the cymba concha allows for the wearable device to co-exist with other in-ear devices such as hearing aids, wired in-ear headphones, or wireless in-ear headphones. In some embodiments, the in-ear device is configured to monitor the subject for at least about 24 hours continuously. In some embodiments, the in-ear device is configured to monitor the subject for at least about 72 hours continuously. In some embodiments, the micro energy storage bank comprises a supercapacitor or a micro battery. In some embodiments, the micro energy storage bank has a maximum capacity of no more than 1 milli-Watt-hour (mWh). In some embodiments, the micro energy storage bank has a maximum capacity of no more than 10 milli-Watt-hour (mWh). In some embodiments, the energy harvesting element compromises a photovoltaic cell configured to harvest energy from natural daylight, interior lighting, infrared emitters, or a combination thereof. In some embodiments, the energy harvesting element comprises a RF antenna configured to harvest energy from the environment of the device. In some embodiments, the energy harvesting element comprises a thermoelectric generator configured to harvest energy from body heat of the subject. In some embodiments, the energy harvesting element comprises a piezoelectric material configured to harvest energy from motion of the subject. In some embodiments, the in-ear device further comprises a logic element performing a charge management protocol comprising: monitoring the charge of the micro energy storage bank; when the charge is below a predetermined threshold, allowing the energy harvesting element to charge the micro energy storage bank; and when the charge is above a second predetermined threshold, allowing the micro energy storage bank to power operation of the biometric sensor, the movement sensor, or the wireless communications transceiver. In some embodiments, the in-ear device further comprises a logic element performing a state management protocol comprising: maintaining the device in a sleep state, wherein the micro energy storage bank is charged; shifting the device to a first wake state intermittently, at a predefined interval, wherein the micro energy storage bank powers operation of the biometric sensor, the movement sensor, and the wireless communications transceiver to perform synchronous monitoring of the subject; and shifting the device to a second wake state as a response to the at least one biometric parameter, the at least one activity parameter, or both, wherein the micro energy storage bank powers operation of the biometric sensor, the movement sensor, and the wireless communications transceiver to perform asynchronous monitoring of the subject.

In some embodiments, the predefined interval is between about 1 minute to about 30 minutes. In some embodiments, the predefined interval is between about 1 minute to about 2 minutes, about 1 minute to about 5 minutes, about 1 minute to about 10 minutes, about 1 minute to about 15 minutes, about 1 minute to about 20 minutes, about 1 minute to about 25 minutes, about 1 minute to about 30 minutes, about 2 minutes to about 5 minutes, about 2 minutes to about 10 minutes, about 2 minutes to about 15 minutes, about 2 minutes to about 20 minutes, about 2 minutes to about 25 minutes, about 2 minutes to about 30 minutes, about 5 minutes to about 10 minutes, about 5 minutes to about 15 minutes, about 5 minutes to about 20 minutes, about 5 minutes to about 25 minutes, about 5 minutes to about 30 minutes, about 10 minutes to about 15 minutes, about 10 minutes to about 20 minutes, about 10 minutes to about 25 minutes, about 10 minutes to about 30 minutes, about 15 minutes to about 20 minutes, about 15 minutes to about 25 minutes, about 15 minutes to about 30 minutes, about 20 minutes to about 25 minutes, about 20 minutes to about 30 minutes, or about 25 minutes to about 30 minutes, including increments therein. In some embodiments, the predefined interval is between about 1 minute, about 2 minutes, about 5 minutes, about 10 minutes, about 15 minutes, about 20 minutes, about 25 minutes, or about 30 minutes. In some embodiments, the predefined interval is between at least about 1 minute, about 2 minutes, about 5 minutes, about 10 minutes, about 15 minutes, about 20 minutes, or about 25 minutes. In some embodiments, the predefined interval is between at most about 2 minutes, about 5 minutes, about 10 minutes, about 15 minutes, about 20 minutes, about 25 minutes, or about 30 minutes.

In some embodiments, the state management further comprises returning the device to the sleep state after performing the synchronous or asynchronous monitoring of the subject for a monitoring period.

In some embodiments, the monitoring period is between about 5 seconds to about 120 seconds. In some embodiments, the monitoring period is between about 5 seconds to about 10 seconds, about 5 seconds to about 20 seconds, about 5 seconds to about 30 seconds, about 5 seconds to about 40 seconds, about 5 seconds to about 50 seconds, about 5 seconds to about 60 seconds, about 5 seconds to about 70 seconds, about 5 seconds to about 80 seconds, about 5 seconds to about 100 seconds, about 5 seconds to about 110 seconds, about 5 seconds to about 120 seconds, about 10 seconds to about 20 seconds, about 10 seconds to about 30 seconds, about 10 seconds to about 40 seconds, about 10 seconds to about 50 seconds, about 10 seconds to about 60 seconds, about 10 seconds to about 70 seconds, about 10 seconds to about 80 seconds, about 10 seconds to about 100 seconds, about 10 seconds to about 110 seconds, about 10 seconds to about 120 seconds, about 20 seconds to about 30 seconds, about 20 seconds to about 40 seconds, about 20 seconds to about 50 seconds, about 20 seconds to about 60 seconds, about 20 seconds to about 70 seconds, about 20 seconds to about 80 seconds, about 20 seconds to about 100 seconds, about 20 seconds to about 110 seconds, about 20 seconds to about 120 seconds, about 30 seconds to about 40 seconds, about 30 seconds to about 50 seconds, about 30 seconds to about 60 seconds, about 30 seconds to about 70 seconds, about 30 seconds to about 80 seconds, about 30 seconds to about 100 seconds, about 30 seconds to about 110 seconds, about 30 seconds to about 120 seconds, about 40 seconds to about 50 seconds, about 40 seconds to about 60 seconds, about 40 seconds to about 70 seconds, about 40 seconds to about 80 seconds, about 40 seconds to about 100 seconds, about 40 seconds to about 110 seconds, about 40 seconds to about 120 seconds, about 50 seconds to about 60 seconds, about 50 seconds to about 70 seconds, about 50 seconds to about 80 seconds, about 50 seconds to about 100 seconds, about 50 seconds to about 110 seconds, about 50 seconds to about 120 seconds, about 60 seconds to about 70 seconds, about 60 seconds to about 80 seconds, about 60 seconds to about 100 seconds, about 60 seconds to about 110 seconds, about 60 seconds to about 120 seconds, about 70 seconds to about 80 seconds, about 70 seconds to about 100 seconds, about 70 seconds to about 110 seconds, about 70 seconds to about 120 seconds, about 80 seconds to about 100 seconds, about 80 seconds to about 110 seconds, about 80 seconds to about 120 seconds, about 100 seconds to about 110 seconds, about 100 seconds to about 120 seconds, or about 110 seconds to about 120 seconds, including increments therein. In some embodiments, the monitoring period is between about 5 seconds, about 10 seconds, about 20 seconds, about 30 seconds, about 40 seconds, about 50 seconds, about 60 seconds, about 70 seconds, about 80 seconds, about 100 seconds, about 110 seconds, or about 120 seconds. In some embodiments, the monitoring period is between at least about 5 seconds, about 10 seconds, about 20 seconds, about 30 seconds, about 40 seconds, about 50 seconds, about 60 seconds, about 70 seconds, about 80 seconds, about 100 seconds, or about 110 seconds. In some embodiments, the monitoring period is between at most about 10 seconds, about 20 seconds, about 30 seconds, about 40 seconds, about 50 seconds, about 60 seconds, about 70 seconds, about 80 seconds, about 100 seconds, about 110 seconds, or about 120 seconds.

In some embodiments, in the first wake state or the second wake state, the power consumption of the device is more than the power output of the energy harvesting element.

In some embodiments, in the first wake state or the second wake state, the biometric sensor monitors the at least one biometric parameter of the subject at a rate of between about 1 Hz to about 200 Hz. In some embodiments, in the first wake state or the second wake state, the biometric sensor monitors the at least one biometric parameter of the subject at a rate of between about 1 Hz to about 10 Hz, about 1 Hz to about 50 Hz, about 1 Hz to about 100 Hz, about 1 Hz to about 150 Hz, about 1 Hz to about 200 Hz, about 10 Hz to about 50 Hz, about 10 Hz to about 100 Hz, about 10 Hz to about 150 Hz, about 10 Hz to about 200 Hz, about 50 Hz to about 100 Hz, about 50 Hz to about 150 Hz, about 50 Hz to about 200 Hz, about 100 Hz to about 150 Hz, about 100 Hz to about 200 Hz, or about 150 Hz to about 200 Hz, including increments therein. In some embodiments, in the first wake state or the second wake state, the biometric sensor monitors the at least one biometric parameter of the subject at a rate of between about 1 Hz, about 10 Hz, about 50 Hz, about 100 Hz, about 150 Hz, or about 200 Hz. In some embodiments, in the first wake state or the second wake state, the biometric sensor monitors the at least one biometric parameter of the subject at a rate of between at least about 1 Hz, about 10 Hz, about 50 Hz, about 100 Hz, or about 150 Hz. In some embodiments, in the first wake state or the second wake state, the biometric sensor monitors the at least one biometric parameter of the subject at a rate of between at most about 10 Hz, about 50 Hz, about 100 Hz, about 150 Hz, or about 200 Hz.

In some embodiments, in the first wake state or the second wake state, the movement sensor monitors the at least one activity parameter of the subject at a rate of between about 1 Hz to about 200 Hz. In some embodiments, in the first wake state or the second wake state, the movement sensor monitors the at least one activity parameter of the subject at a rate of between about 1 Hz to about 10 Hz, about 1 Hz to about 50 Hz, about 1 Hz to about 100 Hz, about 1 Hz to about 150 Hz, about 1 Hz to about 200 Hz, about 10 Hz to about 50 Hz, about 10 Hz to about 100 Hz, about 10 Hz to about 150 Hz, about 10 Hz to about 200 Hz, about 50 Hz to about 100 Hz, about 50 Hz to about 150 Hz, about 50 Hz to about 200 Hz, about 100 Hz to about 150 Hz, about 100 Hz to about 200 Hz, or about 150 Hz to about 200 Hz, including increments therein. In some embodiments, in the first wake state or the second wake state, the movement sensor monitors the at least one activity parameter of the subject at a rate of between about 1 Hz, about 10 Hz, about 50 Hz, about 100 Hz, about 150 Hz, or about 200 Hz. In some embodiments, in the first wake state or the second wake state, the movement sensor monitors the at least one activity parameter of the subject at a rate of between at least about 1 Hz, about 10 Hz, about 50 Hz, about 100 Hz, or about 150 Hz. In some embodiments, in the first wake state or the second wake state, the movement sensor monitors the at least one activity parameter of the subject at a rate of between at most about 10 Hz, about 50 Hz, about 100 Hz, about 150 Hz, or about 200 Hz.

In some embodiments, the attachment mechanism is a biocompatible adhesive. In some embodiments, the attachment mechanism is one or more elastomeric wings with a durometer less than 80 Shore A, configured to extend into the triangular fossa while pressing into the helix, and/or to extend into the concha cavum while pressing into the antihelix or antitragus. In some embodiments, the attachment mechanism is one or more elastomeric clips with a durometer less than 80 Shore A, configured to clip onto the helix and/or antihelix of the subject. In some embodiments, the attachment mechanism is one or more elastomeric rough surface finishes with a durometer less than 80 Shore A, configured to maximize the presence of Van Der Waals forces. In some embodiments, the attachment mechanism is one or more elastomeric suction cups with a durometer less than 80 Shore A, configured to induce a negative pressure to adhere into the cymba concha of the subject. In some embodiments, the attachment mechanism is a set of elastomeric appendages with a durometer less than 80 Shore A, configured to maximize friction by pressing into the helix, antihelix, and/or the cymba concha of the subject. In some embodiments, the attachment mechanism is a custom molded elastomer configured to lock into the subject's unique concha morphology. In some embodiments, the attachment mechanism is a custom 3D print designed to lock into the subject's unique concha morphology.

In some embodiments, the device has a longest dimension of about 6 mm to about 30 mm. In some embodiments, the device has a longest dimension of about 6 mm to about 8 mm, about 6 mm to about 10 mm, about 6 mm to about 12 mm, about 6 mm to about 15 mm, about 6 mm to about 20 mm, about 6 mm to about 25 mm, about 6 mm to about 30 mm, about 8 mm to about 10 mm, about 8 mm to about 12 mm, about 8 mm to about 15 mm, about 8 mm to about 20 mm, about 8 mm to about 25 mm, about 8 mm to about 30 mm, about 10 mm to about 12 mm, about 10 mm to about 15 mm, about 10 mm to about 20 mm, about 10 mm to about 25 mm, about 10 mm to about 30 mm, about 12 mm to about 15 mm, about 12 mm to about 20 mm, about 12 mm to about 25 mm, about 12 mm to about 30 mm, about 15 mm to about 20 mm, about 15 mm to about 25 mm, about 15 mm to about 30 mm, about 20 mm to about 25 mm, about 20 mm to about 30 mm, or about 25 mm to about 30 mm, including increments therein. In some embodiments, the device has a longest dimension of about 6 mm, about 8 mm, about 10 mm, about 12 mm, about 15 mm, about 20 mm, about 25 mm, or about 30 mm. In some embodiments, the device has a longest dimension of at least about 6 mm, about 8 mm, about 10 mm, about 12 mm, about 15 mm, about 20 mm, or about 25 mm. In some embodiments, the device has a longest dimension of at most about 8 mm, about 10 mm, about 12 mm, about 15 mm, about 20 mm, about 25 mm, or about 30 mm.

In some embodiments, the device is adapted to attach or anchor to the auricle of the subject at a cymba concha, cavum concha, scapha, triangular fossa, antitragus, anti-helix, or inner surface of a helix of the subject. In some embodiments, the biometric sensor comprises an optical sensor. In some embodiments, the optical sensor comprises a photoplethysmography (PPG) sensor. In some embodiments, the at least one biometric parameter of the subject comprises cerebral blood flow. In some embodiments, the at least one biometric parameter of the subject comprises blood pressure. In some embodiments, the at least one biometric parameter of the subject comprises blood volume. In some embodiments, the at least one biometric parameter of the subject comprises heart rate. In some embodiments, the at least one biometric parameter of the subject comprises heart rate variability. In some embodiments, the at least one biometric parameter of the subject comprises blood oxygenation. In some embodiments, the movement sensor comprises at least one accelerometer. In some embodiments, the at least one activity parameter of the subject comprises an activity level. In some embodiments, the at least one activity parameter of the subject comprises a body posture or a change in body posture. In some embodiments, the wireless communications transceiver utilizes a Near-Field Communication (NFC) protocol, Bluetooth, Bluetooth Low Energy, LoRa, or Wi-Fi. In some embodiments, the wireless communications transceiver is configured to send data to an external device and receive data from the external device. In some embodiments, the external device comprises a local base station, a mobile device of the subject, or at least one server. In some embodiments, the in-ear device further comprises a temperature sensor. In some embodiments, the at least one biometric parameter of the subject comprises temperature. In some embodiments, the in-ear device is configured for long-term, continuous monitoring of orthostatic hypotension in the subject. In some embodiments, the in-ear device is configured for long-term, continuous monitoring of hypertension in the subject. In some embodiments, the in-ear device further comprises a microcontroller configured to analyze the at least one biometric parameter and the at least one activity parameter. In some embodiments, the analysis comprises application of one or more artificial neural networks (ANNs). In some embodiments, the one or more ANNs are configured to detect or predict one or more of: poor cerebral blood flow, poor blood pressure, presyncope, syncope and a fall event. In some embodiments, the one or more ANNs are configured to infer systolic and diastolic blood pressure from the at least one biometric sensor or activity sensor. In some embodiments, the analysis comprises identification of physiological trends. In some embodiments, the physiological trends comprise trends in one or more of: cerebral blood flow, blood pressure, blood volume, heart rate, heart rate variability, blood oxygenation, and activity level. In some embodiments, the physiological trends are intraday and interday trends.

Another aspect provided herein is a system for long-term, continuous monitoring of a subject, the system comprising an in-ear device and a local base station: the in-ear device comprising: a biometric sensor configured to monitor at least one biometric parameter of the subject; a movement sensor configured to monitor at least one activity parameter of the subject; a micro energy storage bank; an energy harvesting element configured to charge the micro energy storage bank; and a wireless communications transceiver configured to send data to the base station and receive data from the base station; and an attachment mechanism for securing the device to the auricle of the subject; the local base station comprising: a wireless communications transceiver configured to send data to the in-ear device and receive data from the in-ear device; and a network interface configured to provide connectivity to a computer network.

In some embodiments, the local base station further comprises a wireless power transmitter (WPT) comprising an RF energy transmission antenna. In some embodiments, the local base station further comprises a wireless power transmitter (WPT) comprising infrared light emitters. In some embodiments, the infrared light emitters comprise infrared light-emitting diodes (LEDs). In some embodiments, the local base station further comprises one or more processors configured to transmit an alert via one or more of: SMS, MMS, email, telephone, voice mail, and social media. In some embodiments, the local base station further comprises an acoustic transducer for broadcasting audio messages. In some embodiments, the local base station further comprises a screen for displaying biometric information and notifications. In some embodiments, the network interface utilizes one or more of: Wi-Fi, 4G, LTE, and 5G. In some embodiments, the computer network comprises the internet.

Another aspect provided herein is a platform for long-term, continuous monitoring of a subject, the platform comprising an in-ear device, a local base station, and a cloud computing back-end: the in-ear device comprising: and an attachment mechanism for attaching the device to an auricle of the subject; a biometric sensor configured to monitor at least one biometric parameter of the subject; a movement sensor configured to monitor at least one activity parameter of the subject; a micro energy storage bank; an energy harvesting element configured to charge the micro energy storage bank; and a wireless communications transceiver configured to send biometric and activity data of the subject to the base station and receive data from the base station; the local base station comprising: a wireless communications transceiver configured to receive the biometric and activity data of the subject from the in-ear device and send data to the in-ear device; and a network interface configured to provide connectivity to the cloud computing back-end; and a cloud computing back-end comprising a module configured to store and analyze the biometric and activity data of the subject to identify physiological trends and provide biometric feedback or behavioral coaching recommendations.

In some embodiments, the local base station further comprises one or more processors configured to transmit an alert via one or more of: SMS, MMS, email, telephone, voice mail, and social media. In some embodiments, the network interface utilizes one or more of: Wi-Fi, 4G, LTE, and 5G. In some embodiments, the computer network comprises the internet. In some embodiments, the platform is configured for long-term, continuous monitoring of cerebral blood flow in the subject. In some embodiments, the platform is configured for long-term, continuous monitoring of cerebral blood flow in the subject. In some embodiments, the physiological trends comprise intraday and interday cerebral blood flow trends. In some embodiments, the behavioral coaching recommendations pertain to prevention of insufficient cerebral blood flow to avoid a fall. In some embodiments, the platform is configured for long-term, continuous monitoring of hypertension in the subject. In some embodiments, the physiological trends comprise intraday and interday blood pressure trends. In some embodiments, the behavioral coaching recommendations pertain to keeping the blood pressure within a safe range. In some embodiments, the platform is configured for long-term continuous monitoring as a consumer wellness/fitness wearable, providing biometric feedback such as hydration levels and heart rate variability. In some embodiments, the platform is configured to monitor and manage precipitating factors of stroke and dementia, such as unstable cerebral blood flow. In some embodiments, the platform is configured to manage syncope-related hospital emergency room visits through biometric data driven triage. In some embodiments, the platform is configured to manage cerebral oxygenation and hemodynamics in hospital Intensive Care Units (ICU) or Operating Rooms (OR). In some embodiments, the platform is configured as a high adherence, forget-it's-there, remote patient monitor (RPM) for long term care facilities. In some embodiments, the cloud computing back-end further comprises a module configured to provide a healthcare provider portal application allowing access to real time and historical data and trends for one or more subjects. In some embodiments, the cloud computing back-end further comprises a module configured to provide a subject health portal application allowing access to real time and historical data and trends for the subject. In some embodiments, the analysis comprises application of one or more artificial neural networks (ANNs). In some embodiments, the one or more ANNs are configured to predict insufficient cerebral blood flow or blood pressure events.

Another aspect provided herein is a method of performing long-term, continuous monitoring of a subject with an in-ear device comprising a biometric sensor, a movement sensor, a micro energy storage bank, an energy harvesting element, and a wireless communications transceiver, the method comprising: maintaining the device in a sleep state, wherein the micro energy storage bank is charged by the energy harvesting element; shifting the device to a first wake state intermittently, at a predefined interval, wherein the micro energy storage bank powers operation of the biometric sensor, the movement sensor, and the wireless communications transceiver to perform synchronous monitoring of at least one biometric parameter of the subject and at least one activity parameter of the subject; and shifting the device to a second wake state based on the at least one biometric parameter, the at least one activity parameter, or both, wherein the micro energy storage bank powers operation of the biometric sensor, the movement sensor, and the wireless communications transceiver to perform asynchronous monitoring of the at least one biometric parameter of the subject and the at least one activity parameter of the subject of the subject.

In some embodiments, the predefined interval is between about 1 minute to about 30 minutes. In some embodiments, the predefined interval is between about 1 minute to about 2 minutes, about 1 minute to about 5 minutes, about 1 minute to about 10 minutes, about 1 minute to about 15 minutes, about 1 minute to about 20 minutes, about 1 minute to about 25 minutes, about 1 minute to about 30 minutes, about 2 minutes to about 5 minutes, about 2 minutes to about 10 minutes, about 2 minutes to about 15 minutes, about 2 minutes to about 20 minutes, about 2 minutes to about 25 minutes, about 2 minutes to about 30 minutes, about 5 minutes to about 10 minutes, about 5 minutes to about 15 minutes, about 5 minutes to about 20 minutes, about 5 minutes to about 25 minutes, about 5 minutes to about 30 minutes, about 10 minutes to about 15 minutes, about 10 minutes to about 20 minutes, about 10 minutes to about 25 minutes, about 10 minutes to about 30 minutes, about 15 minutes to about 20 minutes, about 15 minutes to about 25 minutes, about 15 minutes to about 30 minutes, about 20 minutes to about 25 minutes, about 20 minutes to about 30 minutes, or about 25 minutes to about 30 minutes, including increments therein. In some embodiments, the predefined interval is between about 1 minute, about 2 minutes, about 5 minutes, about 10 minutes, about 15 minutes, about 20 minutes, about 25 minutes, or about 30 minutes. In some embodiments, the predefined interval is between at least about 1 minute, about 2 minutes, about 5 minutes, about 10 minutes, about 15 minutes, about 20 minutes, or about 25 minutes. In some embodiments, the predefined interval is between at most about 2 minutes, about 5 minutes, about 10 minutes, about 15 minutes, about 20 minutes, about 25 minutes, or about 30 minutes.

In some embodiments, in the first wake state or the second wake state, the power consumption of the device is more than the power output of the energy harvesting element.

In some embodiments, in the first wake state or the second wake state, the biometric sensor monitors the at least one biometric parameter of the subject at a rate of between about 1 Hz to about 200 Hz. In some embodiments, in the first wake state or the second wake state, the biometric sensor monitors the at least one biometric parameter of the subject at a rate of between about 1 Hz to about 10 Hz, about 1 Hz to about 50 Hz, about 1 Hz to about 100 Hz, about 1 Hz to about 150 Hz, about 1 Hz to about 200 Hz, about 10 Hz to about 50 Hz, about 10 Hz to about 100 Hz, about 10 Hz to about 150 Hz, about 10 Hz to about 200 Hz, about 50 Hz to about 100 Hz, about 50 Hz to about 150 Hz, about 50 Hz to about 200 Hz, about 100 Hz to about 150 Hz, about 100 Hz to about 200 Hz, or about 150 Hz to about 200 Hz, including increments therein. In some embodiments, in the first wake state or the second wake state, the biometric sensor monitors the at least one biometric parameter of the subject at a rate of between about 1 Hz, about 10 Hz, about 50 Hz, about 100 Hz, about 150 Hz, or about 200 Hz. In some embodiments, in the first wake state or the second wake state, the biometric sensor monitors the at least one biometric parameter of the subject at a rate of between at least about 1 Hz, about 10 Hz, about 50 Hz, about 100 Hz, or about 150 Hz. In some embodiments, in the first wake state or the second wake state, the biometric sensor monitors the at least one biometric parameter of the subject at a rate of between at most about 10 Hz, about 50 Hz, about 100 Hz, about 150 Hz, or about 200 Hz.

In some embodiments, in the first wake state or the second wake state, the movement sensor monitors the at least one activity parameter of the subject at a rate of between about 1 Hz to about 200 Hz. In some embodiments, in the first wake state or the second wake state, the movement sensor monitors the at least one activity parameter of the subject at a rate of between about 1 Hz to about 10 Hz, about 1 Hz to about 50 Hz, about 1 Hz to about 100 Hz, about 1 Hz to about 150 Hz, about 1 Hz to about 200 Hz, about 10 Hz to about 50 Hz, about 10 Hz to about 100 Hz, about 10 Hz to about 150 Hz, about 10 Hz to about 200 Hz, about 50 Hz to about 100 Hz, about 50 Hz to about 150 Hz, about 50 Hz to about 200 Hz, about 100 Hz to about 150 Hz, about 100 Hz to about 200 Hz, or about 150 Hz to about 200 Hz, including increments therein. In some embodiments, in the first wake state or the second wake state, the movement sensor monitors the at least one activity parameter of the subject at a rate of between about 1 Hz, about 10 Hz, about 50 Hz, about 100 Hz, about 150 Hz, or about 200 Hz. In some embodiments, in the first wake state or the second wake state, the movement sensor monitors the at least one activity parameter of the subject at a rate of between at least about 1 Hz, about 10 Hz, about 50 Hz, about 100 Hz, or about 150 Hz. In some embodiments, in the first wake state or the second wake state, the movement sensor monitors the at least one activity parameter of the subject at a rate of between at most about 10 Hz, about 50 Hz, about 100 Hz, about 150 Hz, or about 200 Hz.

In some embodiments, the at least one biometric parameter of the subject comprises one or more of: cerebral blood flow, blood pressure, blood volume, heart rate, heart rate variability, and blood oxygenation. In some embodiments, the at least one activity parameter of the subject comprises one or more of: activity level, body posture, and change in body posture. In some embodiments, the method comprises monitoring the charge of the micro energy storage bank, wherein, when the charge is below a predetermined threshold, maintaining the device in the sleep state, and wherein, when the charge is above a second predetermined threshold, allowing the device to shift to the first wake state or the second wake state. In some embodiments, the micro energy storage bank is charged by the energy harvesting element utilizing one or more of: a photovoltaic cell configured to harvest energy from the environment of the subject, a RF harvesting antenna configured to harvest energy from the environment of the subject, a thermoelectric generator configured to harvest energy from body heat of the subject, and a piezoelectric material configured to harvest energy from motion of the subject.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows a diagram of the components of an exemplary in-ear device; per an embodiment herein;

FIG. 2 shows an illustration of an exemplary in-ear device; per an embodiment herein;

FIG. 3 shows an image of an exemplary in-ear device; per an embodiment herein;

FIG. 4A shows an illustration of an exemplary in-ear device with a first attachment mechanism; per an embodiment herein;

FIG. 4B shows an illustration of an exemplary in-ear device with a second attachment mechanism; per an embodiment herein;

FIG. 4C shows an illustration of an exemplary in-ear device with a third attachment mechanism; per an embodiment herein;

FIG. 4D shows an illustration of an exemplary in-ear device with a fourth attachment mechanism; per an embodiment herein;

FIG. 4E shows an illustration of an exemplary in-ear device with a fifth attachment mechanism; per an embodiment herein;

FIG. 4F shows an illustration of an exemplary in-ear device with a sixth attachment mechanism; per an embodiment herein;

FIG. 4G shows an illustration of an exemplary in-ear device with a seventh attachment mechanism; per an embodiment herein;

FIG. 5 shows a flowchart of the energy and data transfer in an exemplary in-ear system; per an embodiment herein;

FIG. 6 shows an illustration of an exemplary graphical user interface (GUI) for displaying intraday cerebral blood flow changes, blood pressure, heart rate, and blood oxygenation by an in-ear device mechanism; per an embodiment herein;

FIG. 7 shows a cerebral blood flow vs time graph with consciousness warnings and alerts; per an embodiment herein;

FIG. 8 shows a PPG measured pressure vs time graph with labeled systolic peak, dichrotic notch, and diastolic peak points; per an embodiment herein;

FIG. 9 shows an exemplary graph of absorption of the skin and corresponding DC and AC levels; per an embodiment herein;

FIG. 10 shows a non-limiting example of a computing device; in this case, a device with one or more processors, memory, storage, and a network interface; per an embodiment herein;

FIG. 11 shows a non-limiting example of a web/mobile application provision system; in this case, a system providing browser-based and/or native mobile user interfaces; per an embodiment herein; and

FIG. 12 shows a non-limiting example of a cloud-based web/mobile application provision system; in this case, a system comprising an elastically load balanced, auto-scaling web server and application server resources as well synchronously replicated databases; per an embodiment herein;

FIG. 13 shows another flowchart of the energy and data transfer in an exemplary in-ear system; per an embodiment herein; and

FIG. 14 shows a flowchart of exemplary device states of the in-ear device; per an embodiment herein.

### DETAILED DESCRIPTION

Provided herein are devices, systems, platforms and methods for long-term, continuous monitoring of a biometric parameter of a subject. In some embodiments, the continuous monitoring provides an alert indicating poor cerebral blood flow, poor blood pressure, presyncope, syncope, or any combination thereof.

In-Ear Devices

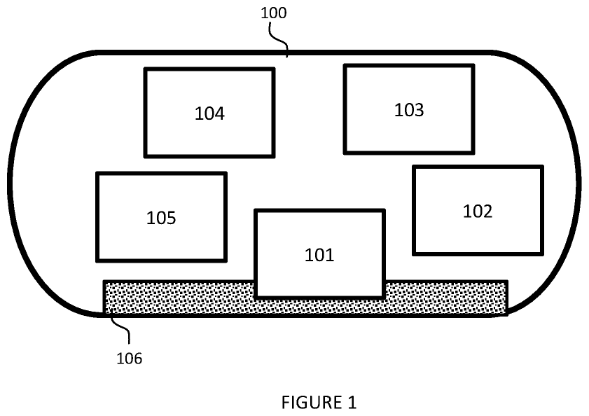

Provided herein, per FIGS. 1-4 are exemplary in-ear devices 100 for long-term, continuous monitoring of a subject. In some embodiments, the in-ear device 100 comprises a biometric sensor 101, a movement sensor 102, a micro energy storage bank 103, an energy harvesting element 104, a wireless communications transceiver 105, and an attachment mechanism 106.

In some embodiments, the one or more biometric sensors targets the Cymba Concha, enabling excellent signal quality due to proximity to branches of the Posterior Auricular Artery. In some embodiments, the posterior auricular artery climbs up the back of the ear, perforates through the ear cartilage to the front of the ear, and travels across the Cymba Concha. In some embodiments, the biometric sensors herein target this branch of the posterior auricular artery for improved sensing. In some embodiments, targeting this branch of the posterior auricular artery increases photoplethysmography (PPG) quality. The Cymba Concha location also enables the device to be worn concurrently with hearing aids and music earbuds, which is key to allowing long-term continuous monitoring. In some embodiments, the in-ear device 100 is configured for long-term, continuous monitoring of cerebral blood flow in the subject. In some embodiments, the in-ear device 100 is configured for long-term, continuous monitoring of blood pressure in the subject. In some embodiments, the control systems, methods, and/or parameters of the biometric sensor 101, the movement sensor 102, the micro energy storage bank 103, the energy harvesting element 104, the attachment mechanism 106 herein, or any combination thereof enables the long-term, continuous monitoring of blood pressure in the subject.

In some embodiments, the continuous monitoring comprises monitoring the subject for 99%, 98% 97%, 96%, 95%, 94%, 93%, 92%, 91%, 90% continuously over a set period of time.

In some embodiments, the continuous monitoring comprises monitoring the subject at least once every 1, 2, 3, 4, 5, 6, 7, 8, 9, 10, 12, 15, 20, 25, 30 minutes or more, including increments therein, over a set period of time. In some embodiments, the continuous monitoring comprises monitoring the subject during at least a portion of a subject's standing transition events, over a set period of time. In some embodiments, the continuous monitoring comprises monitoring the subject during at least about 90% of a subject's standing transition events, over a set period of time. In some embodiments, the continuous monitoring comprises monitoring the subject for at least 1, 2, 3, 4, 5, 6, 7, 8, 9 or more minutes after at least a portion of a subject's standing transition events, over a set period of time. In some embodiments, the continuous monitoring comprises monitoring the subject for at least 1, 2, 3, 4, 5, 6, 7, 8, 9 or more minutes after at least about 90% of a subject's standing transition events, over a set period of time. In some embodiments, the continuous monitoring comprises monitoring the subject for at least 5, 10, 15, 20, 25, 30, 35, 40, 45, 50, 55, 60 or more minutes within a set period of time. In some embodiments, the micro energy storage bank will last up to 30 minutes. In some embodiments, the micro energy storage bank is able to perform multiple levels of functionality with low energy storage because of continuous energy harvesting top-off that occurs while the device is being worn. In some embodiments, the micro energy storage bank is charged more than once per day. In some embodiments, the micro energy storage device is continuously charged throughout the day by continuous energy harvesting.

In some embodiments, the set period of time is at least about 12 hours, 16 hours, 20 hours, 24 hours, 30 hours, 34 hours, 38 hours, 42 hours, or 48 hours or more continuously, including increments therein. In some embodiments, the set period of time is at least about 48 hours, 52 hours, 56 hours, 60 hours, 64 hours, 68 hours, 72 hours, 76 hours, 80 hours, 84 hours, 88 hours or more continuously, including increments therein. In some embodiments, the set period of time is at least about 1, 2, 3, 4, 5, 6, 7, 8, 9, 10 or more days.

In some embodiments, the in-ear device 100 is configured to continuously monitor the subject for at least about 12 hours, 16 hours, 20 hours, 24 hours, 30 hours, 34 hours, 38 hours, 42 hours, or 48 hours or more continuously, including increments therein. In some embodiments, the in-ear device 100 is configured to continuously monitor the subject for at least about 48 hours, 52 hours, 56 hours, 60 hours, 64 hours, 68 hours, 72 hours, 76 hours, 80 hours, 84 hours, 88 hours or more continuously, including increments therein. In some embodiments, the in-ear device 100 is configured to continuously monitor the subject for at least about 1, 2, 3, 4, 5, 6, 7, 8, 9, 10 or more days.

In some embodiments, the in-ear device 100 is 15 mm or less in its longest dimension. In some embodiments, the in-ear device 100 is 12 mm or less in its longest dimension. In some embodiments, the size and power management of the biometric sensor 101, the movement sensor 102, the micro energy storage bank 103, the energy harvesting element 104, the attachment mechanism 106 or any combination thereof herein enables the small in-ear device 100 dimensions. The small in-ear device 100 disclosed herein is inconspicuous so as to allow for minimal stigma, and requires nearly zero maintenance upkeep by the user. These characteristics are paramount to reduce usage friction and consequently the patient's adherence to wearing the device.

In some embodiments, the biometric sensor 101 is configured to monitor at least one biometric parameter of the subject. In some embodiments, the biometric sensor 101 comprises an optical sensor. In some embodiments, the optical sensor comprises a photoplethysmography (PPG) sensor. In some embodiments, the photoplethysmography (PPG) sensor comprises a green LED, a red LED, an infrared LED, or any combination thereof. In some embodiments, the photoplethysmography (PPG) sensor comprises a light emitting diode (LED). In some embodiments, the LED has a current of about 31 mA. In some embodiments, the at least one biometric parameter of the subject comprises cerebral blood flow. In some embodiments, the at least one biometric parameter of the subject comprises blood pressure. In some embodiments, the at least one biometric parameter of the subject comprises blood volume. In some embodiments, the at least one biometric parameter of the subject comprises heart rate. In some embodiments, the at least one biometric parameter of the subject comprises heart rate variability. In some embodiments, the at least one biometric parameter of the subject comprises blood oxygenation. In some embodiments, the in-ear device 100 further comprises a temperature sensor. In some embodiments, the at least one biometric parameter of the subject comprises temperature.

In some embodiments, the movement sensor 102 is configured to monitor at least one activity parameter of the subject. In some embodiments, the movement sensor 102 comprises at least one accelerometer. In some embodiments, the movement sensor 102 comprises 2 or 3 accelerometers. In some embodiments, the accelerometer measures acceleration in three or more perpendicular axes. In some embodiments, the at least one activity parameter of the subject comprises an activity level. In some embodiments, the at least one activity parameter of the subject comprises a body posture or a change in body posture.

In some embodiments, the micro energy storage bank 103 comprises a supercapacitor, a micro battery or both. In some embodiments, the micro energy storage bank 103 has a high energy density, a high power density, or both. In some embodiments, the micro energy storage bank 103 has a maximum capacity of no more than about 0.25 milli-Watt-hour (mWh), 0.25 mWh, 0.75 mWh, 1 mWh, 1.25 mWh, 1.5 mWh, or 2 mWh, including increments therein. In some embodiments, the capacity of the micro energy storage bank 103 enables actively measuring and collecting the subject's biometric data for at least about 24 hours continuously. In some embodiments, the capacity of the micro energy storage bank 103 enables actively measuring and collecting the subject's biometric data for at least about 72 hours continuously. In some embodiments, a size of the micro energy storage bank 103 enables the in-ear device 100 size of about 15 mm or less in its longest dimension.

In some embodiments, the energy harvesting element 104 is configured to charge the micro energy storage bank 103. In some embodiments, the energy harvesting element 104 compromises a photovoltaic cell configured to harvest energy from natural daylight, interior lighting, infrared emitters, or a combination thereof. In some embodiments, the energy harvesting element 104 comprises a RF antenna configured to harvest energy from the environment of the in-ear device 100. In some embodiments, the energy harvesting element 104 comprises a thermoelectric generator configured to harvest energy from body heat of the subject. In some embodiments, the energy harvesting element 104 comprises a piezoelectric material configured to harvest energy from motion of the subject. In some embodiments, the use of the energy harvesting element 104 to charge the micro energy storage bank 103 in-ear device 100 enables monitoring of the subject for at least about 24 hours continuously. In some embodiments, the use of the energy harvesting element 104 to charge the micro energy storage bank 103 in-ear device 100 enables monitoring of the subject for at least about 72 hours continuously. In some embodiments, a design and efficiency of the RF antenna, the thermoelectric generator, the piezoelectric material, or any combination thereof enables monitoring of the subject for at least about 24 hours continuously. In some embodiments, a design and efficiency of the RF antenna, the thermoelectric generator, the piezoelectric material, or any combination thereof enables monitoring of the subject for at least about 72 hours continuously.

In some embodiments, the energy harvesting element 104, the micro energy storage bank 103, the RF antenna, the thermoelectric generator, the piezoelectric material, or any combination thereof enable the in-ear device 100 to continuously monitor the subject for at least about 12 hours, 16 hours, 20 hours, 24 hours, 30 hours, 34 hours, 38 hours, 42 hours, or 48 hours or more continuously, including increments therein. In some embodiments, the energy harvesting element 104, the micro energy storage bank 103, the RF antenna, the thermoelectric generator, the piezoelectric material, or any combination thereof enable the in-ear device 100 to monitor the subject for at least about 48 hours, 52 hours, 56 hours, 60 hours, 64 hours, 68 hours, 72 hours, 76 hours, 80 hours, 84 hours, 88 hours or more continuously, including increments therein. In some embodiments, the energy harvesting element 104, the micro energy storage bank 103, the RF antenna, the thermoelectric generator, the piezoelectric material, or any combination thereof enable the in-ear device 100 to monitor the subject for at least about 1, 2, 3, 4, 5, 6, 7, 8, 9, 10 or more days.

In some embodiments, the wireless communications transceiver 105 utilizes a Near-Field Communication (NFC) protocol, Bluetooth, Bluetooth Low Energy, LoRa, or Wi-Fi. In some embodiments, the wireless communications transceiver 105 is configured to send data to an external device and receive data from the external device. In some embodiments, the external device comprises a local base station, a mobile device of the subject, or at least one server.

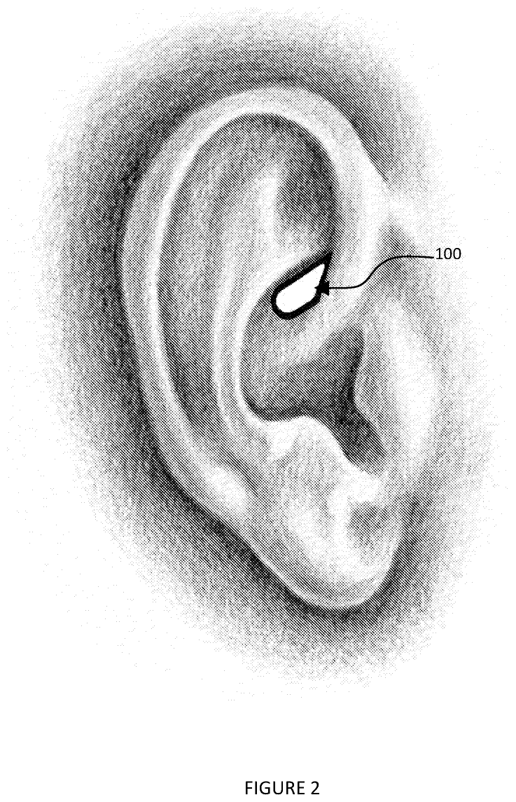

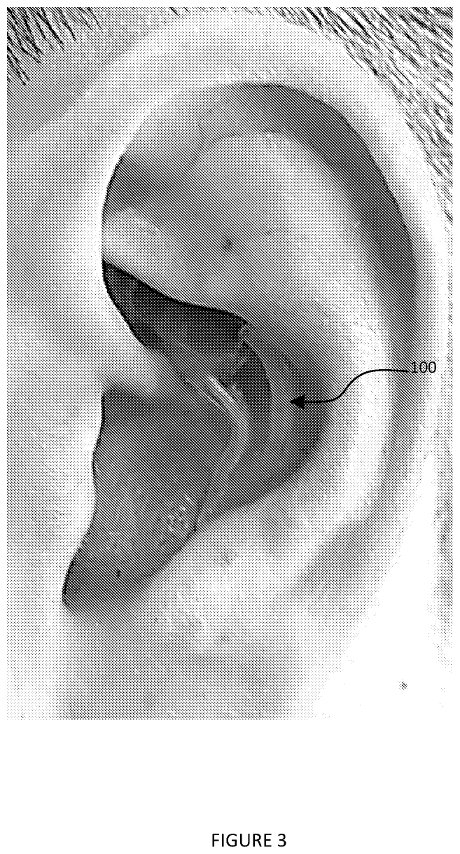

In some embodiments, per FIGS. 2 and 3, the in-ear device 100 is adapted to attach to the auricle of the subject at the cymba concha. Alternatively, in some embodiments, the in-ear device 100 is adapted to attach to the cavum concha, scapha, triangular fossa, anti-helix, or inner surface of a helix of the subject. In some embodiments, the in-ear device 100 is adapted to attach to the auricle of the subject at a cymba concha of the subject. In some embodiments, the attachment mechanism 106 is configured for securing the in-ear device 100 to the auricle of the subject. In some embodiments, the attachment mechanism 106 is configured for securing the in-ear device 100 to the auricle of the subject for at least about 24 hours continuously. In some embodiments, the attachment mechanism 106 is configured for securing the in-ear device 100 to the auricle of the subject for at least about 72 hours continuously.

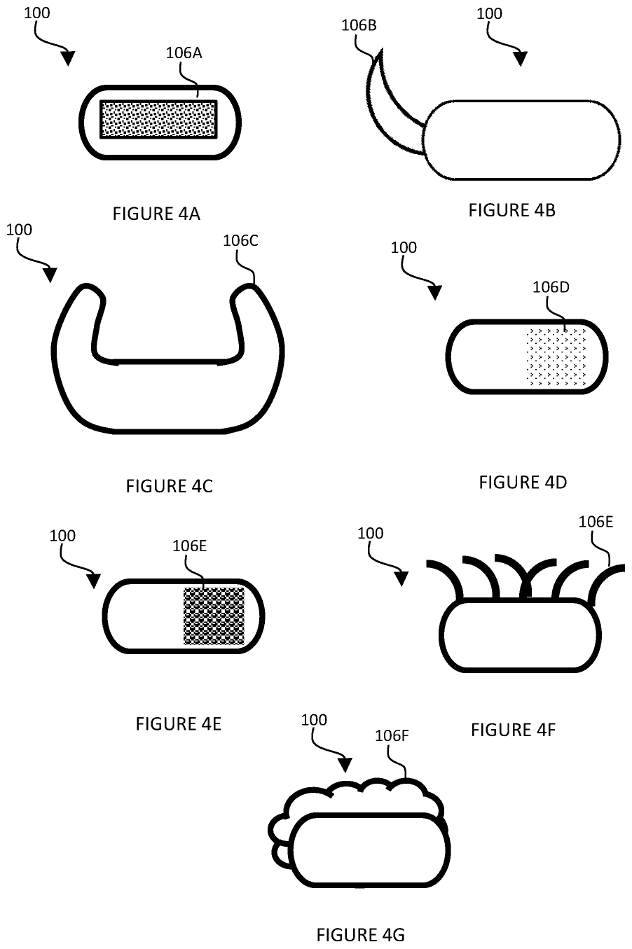

In some embodiments, per FIGS. 1 and 4A, the attachment mechanism 106 is a biocompatible adhesive 106A. In some embodiments, the biocompatible adhesive 106A comprises a natural polymeric adhesive 106A. In some embodiments, the natural polymetric adhesive 106A comprises a fibrin adhesive 106A, a collagen adhesive 106A, a gelati adhesive 106A, an albumin adhesive 106A, a chitosan adhesive 106A, an alginate adhesive 106A, a chondroitin sulfate adhesive 106A, or any combination thereof. In some embodiments, the biocompatible adhesive 106A comprises a synthetic adhesive 106A. In some embodiments, the synthetic adhesive 106A comprises cyanoacrylate, poly ethylene glycol, a dendrimer, polyurethane, polyester, or any combination thereof. In some embodiments, a type of the biocompatible adhesive 106A, an amount of the biocompatible adhesive 106A, or both, is configured for securing the in-ear device 100 to the auricle of the subject for at least about 24 hours continuously. In some embodiments, a type of the biocompatible adhesive 106A, an amount of the biocompatible adhesive 106A, or both, is configured for securing the in-ear device 100 to the auricle of the subject for at least about 72 hours continuously

In some embodiments, per FIG. 4B, the attachment mechanism 106 comprises one or more elastomeric wings 106B. An in-ear device 100 comprising the elastomeric wings 106B is shown in FIG. 3. In some embodiments, the one or more wings 106B is configured to extend into the triangular fossa. In some embodiments, the one or more wings 106B is configured to extend into the triangular fossa while pressing into the helix. In some embodiments, the one or more wings 106B is configured to extend into the concha cavum. In some embodiments, the one or more wings 106B is configured to extend into the concha cavum while pressing into the antihelix or antitragus. In some embodiments, the one or more wings 106B has a durometer less than about 25 Shore A, 38 Shore A, 50 Shore A, 63 Shore A, 75 Shore A, 88 Shore A, or 100 Shore A, including increments therein. In some embodiments, the shape, durometer, or both of the elastomeric wing 106B is configured for securing the in-ear device 100 to the auricle of the subject for at least about 24 hours continuously. In some embodiments, the shape, durometer, or both of the elastomeric wing 106B is configured for securing the in-ear device 100 to the auricle of the subject for at least about 72 hours continuously.

In some embodiments, per FIG. 4C, the attachment mechanism 106 is one or more elastomeric clips 106C. In some embodiments, the one or more elastomeric clips 106C is configured to clip onto the helix of a subject. In some embodiments, the one or more elastomeric clips 106C is configured to clip onto the antihelix of the subject. In some embodiments, the one or more elastomeric clips 106C is configured to clip onto the helix and the antihelix of the subject. In some embodiments, the one or more elastomeric clips 106C have a durometer less than about 40 Shore A, 55 Shore A, 70 Shore A, 85 Shore A, or 100 Shore A, including increments therein. In some embodiments, the shape, durometer, or both of the elastomeric clips 106C is configured for securing the in-ear device 100 to the auricle of the subject for at least about 24 hours continuously. In some embodiments, the shape, durometer, or both of the elastomeric clips 106C is configured for securing the in-ear device 100 to the auricle of the subject for at least about 72 hours continuously.

In some embodiments, per FIG. 4D, the attachment mechanism 106 is one or more elastomeric rough surface finishes 106D. In some embodiments, the one or more elastomeric rough surface finishes 106D is configured to maximize the presence of Van Der Waals forces. In some embodiments, the one or more rough surface finishes 106D have a durometer less than about 40 Shore A, 55 Shore A, 70 Shore A, 85 Shore A, or 100 Shore A, including increments therein. In some embodiments, the shape, durometer, surface finish, or any combination thereof of the elastomeric rough surface is configured for securing the in-ear device 100 to the auricle of the subject for at least about 24 hours continuously. In some embodiments, the shape, durometer, surface finish, or any combination thereof of the elastomeric rough surface is configured for securing the in-ear device 100 to the auricle of the subject for at least about 72 hours continuously.

In some embodiments, per FIG. 4E, the attachment mechanism 106 is one or more elastomeric suction cups 106E. In some embodiments, the elastomeric suction cups 106E are configured to induce a negative pressure to adhere into the concha cymba of the subject. In some embodiments, the elastomeric suction cups 106E have a durometer less than about 40 Shore A, 55 Shore A, 70 Shore A, 85 Shore A, or 100 Shore A, including increments therein. In some embodiments, the shape, durometer, or both of the elastomeric suction cups 106E is configured for securing the in-ear device 100 to the auricle of the subject for at least about 24 hours continuously. In some embodiments, the shape, durometer, or both of the elastomeric suction cups 106E is configured for securing the in-ear device 100 to the auricle of the subject for at least about 72 hours continuously.

In some embodiments, per FIG. 4F, the attachment mechanism 106 is a set of elastomeric appendages 106E with a durometer less than 50 Shore A, configured to maximize friction by pressing into the helix, antihelix, and/or the cymba concha of the subject. In some embodiments, the attachment mechanism 106 is a custom molded elastomer configured to lock into the subject's unique concha morphology. In some embodiments, the attachment mechanism 106 is a custom 3D print designed to lock into the subject's unique concha morphology. In some embodiments, the shape, durometer, or both of the elastomeric appendages 106E is configured for securing the in-ear device 100 to the auricle of the subject for at least about 24 hours continuously. In some embodiments, the shape, durometer, or both of the elastomeric appendages 106E is configured for securing the in-ear device 100 to the auricle of the subject for at least about 72 hours continuously.

In some embodiments, per FIG. 4G, the attachment mechanism 106 is an elastomeric mold 106F with a durometer less than 50 Shore A, configured to maximize friction by pressing into the helix, antihelix, antitragus, and/or the cymba concha of the subject. In some embodiments, the attachment mechanism 106 is a custom molded elastomer configured to lock into the subject's unique concha morphology. In some embodiments, the attachment mechanism 106 is a custom 3D print designed to lock into the subject's unique concha morphology. In some embodiments, the shape, durometer, or both of the elastomeric appendages 106E is configured for securing the in-ear device 100 to the auricle of the subject for at least about 24 hours continuously. In some embodiments, the shape, durometer, or both of the elastomeric appendages 106E is configured for securing the in-ear device 100 to the auricle of the subject for at least about 72 hours continuously. In some embodiments, the device 100 does not comprise the attachment mechanism 106. In some embodiments, the device 100 does not comprise the attachment mechanism 106, wherein the device attaches to the ear of the subject via a mechanical force (i.e. a magnet, a clip, a piercing).

In some embodiments, the in-ear device 100 further comprises a logic element. In some embodiments, the logic element is configured to perform a charge management protocol. In some embodiments, the charge management protocol comprises monitoring the charge of the micro energy storage bank 103; allowing the energy harvesting element 104 to charge the micro energy storage bank 103, and allowing the micro energy storage bank 103 to power operation of the biometric sensor 101, the movement sensor 102, or the wireless communications transceiver 105. In some embodiments, the logic element allows the energy harvesting element 104 to charge the micro energy storage bank 103 when the charge is below a predetermined threshold. In some embodiments, the logic element allows the micro energy storage bank 103 to power operation of the biometric sensor 101, the movement sensor 102, or the wireless communications transceiver 105 when the charge exceeds the is above a second predetermined threshold. In some embodiments, the charge management protocol is configured for increased energy harvesting and decreased energy use, to enable monitoring of the subject for at least about 24 hours continuously. In some embodiments, the charge management protocol is configured for increased energy harvesting and decreased energy use, to enable monitoring of the subject for at least about 72 hours continuously.

In some embodiments, the logic element is configured to perform a state management protocol. In some embodiments, the state management protocol comprises maintaining the in-ear device 100 in a sleep state, shifting the in-ear device 100 to a first wake state, and shifting the in-ear device 100 to a second wake state. In some embodiments, the micro energy storage bank 103 is charged during the sleep state. In some embodiments, the in-ear device 100 is shifted to the first wake state intermittently, at a predefined interval, or both. In some embodiments, the micro energy storage bank 103 powers operation of the biometric sensor 101, the movement sensor 102, and the wireless communications transceiver 105 during the first wake state. In some embodiments, the protocol performs synchronous monitoring of the subject during the first wake state. In some embodiments, the protocol shifts the in-ear device 100 to the second wake state as a response to the at least one biometric parameter, the at least one activity parameter, or both. In some embodiments, the micro energy storage bank 103 powers operation of the biometric sensor 101, the movement sensor 102, and the wireless communications transceiver 105 during the second wake state. In some embodiments, during the second wake state, the protocol performs asynchronous monitoring of the subject. In some embodiments, the state management protocol is configured for increased energy harvesting and decreased energy use, to enable monitoring of the subject for at least about 24 hours continuously. In some embodiments, the state management protocol is configured for increased energy harvesting and decreased energy use, to enable monitoring of the subject for at least about 72 hours continuously.

In some embodiments, the predefined interval is between about 1 minute and 30 minutes. In some embodiments, the state management further comprises returning the in-ear device 100 to the sleep state after performing the synchronous or asynchronous monitoring of the subject for a monitoring period. In some embodiments, the monitoring period is between about 5 seconds and 120 seconds. In some embodiments, in the first wake state or the second wake state, the power consumption of the in-ear device 100 is more than the power output of the energy harvesting element 104. In some embodiments, in the first wake state or the second wake state, the biometric sensor 101 monitors the at least one biometric parameter of the subject at a rate of between about 1 Hz and 200 Hz. In some embodiments, in the first wake state or the second wake state, the movement sensor 102 monitors the at least one activity parameter of the subject at a rate of between about 1 Hz and 200 Hz.

In some embodiments, the sleep state, the first wake state, the second wake state, the predefined interval, the monitoring period, the monitoring rate, or any combination thereof is configured to enable monitoring of the subject for at least about 24 hours continuously. In some embodiments, the sleep state, the first wake state, the second wake state, the predefined interval, the monitoring period, the monitoring rate, or any combination thereof is configured to enable monitoring of the subject for at least about 72 hours continuously.

In some embodiments, the in-ear device 100 further comprises a microcontroller. In some embodiments, the microcontroller is configured to analyze the at least one biometric parameter and the at least one activity parameter. In some embodiments, the analysis comprises application of one or more artificial neural networks (ANNs). In some embodiments, the one or more ANNs are configured to detect or predict one or more of: poor cerebral blood flow, poor blood pressure, presyncope, syncope, and a fall event. In some embodiments, the one or more ANNs are configured to detect or predict one or more of: poor cerebral blood flow, poor blood pressure, presyncope, syncope, and a fall event based on indications within the biometric parameter. In some embodiments, the one or more ANNs are configured to infer systolic and diastolic blood pressure from the at least one biometric sensor 101 or activity sensor. In some embodiments, the analysis comprises identification of physiological trends. In some embodiments, the physiological trends comprise trends in one or more of: cerebral blood flow, blood pressure, blood volume, heart rate, heart rate variability, blood oxygenation, and activity level. In some embodiments, the physiological trends are intraday trends. In some embodiments, the physiological trends are time of day trends.

### Systems for Long-Term Continuous Monitoring of a Subject

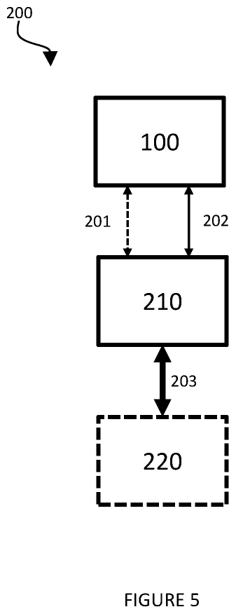

Provided herein, per FIG. 5, is a system 200 for long-term, continuous monitoring of a subject. In some embodiments, the system 200 comprises any embodiment of the in-ear device 100 described herein and a local base station 210.

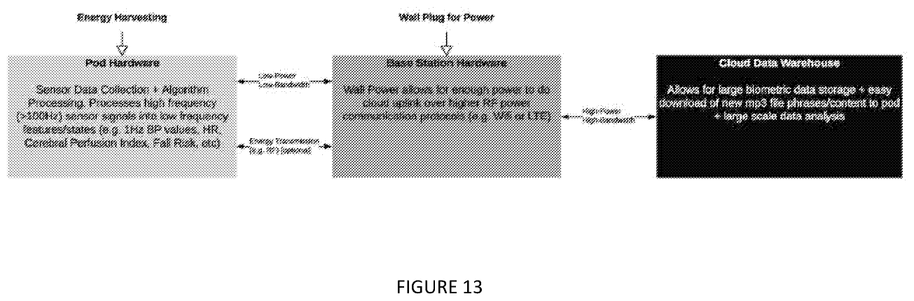

In some embodiments, per FIGS. 5 and 13, the local base station 210 comprises a wireless communications transceiver and a network 220 interface. In some embodiments, the wireless communication transceiver is configured to send a first data 201 to the in-ear device 100 and receive a first data 201 from the in-ear device 100. In some embodiments, the network interface is configured to provide connectivity to a computer network 220. In some embodiments, the network interface is configured to transmit a second data 203 to the computer network 220. In some embodiments, the first data 201, the second data 203, or both comprise the biometric parameter, the activity parameter, or both. In some embodiments, the first data 201, the second data 203, or both are based on the biometric parameter, the activity parameter, or both. In some embodiments, a transmission/reception bandwidth of the second data 203 is greater than a transmission/reception bandwidth of the first data 201. In some embodiments, power provided to the local base station 210 by a battery or a wall outlet enables the transmission/reception bandwidth of the second data 203 to be greater than a transmission/reception bandwidth of the first data 201. In some embodiments, the difference between the transmission/reception bandwidth of the second data 203 and the first data 201 reduces the power required by the in-ear device 100 to communicate with the computer network 220. In some embodiments, this reduced power requirement enables monitoring of the subject for at least about 24 hours continuously. In some embodiments, this reduced power requirement enables monitoring of the subject for at least about 72 hours continuously.

In some embodiments, the sleep state, the first wake state, the second wake state, the predefined interval, the monitoring period, the monitoring rate, or any combination thereof is configured to enable monitoring of the subject for at least about 24 hours continuously. In some embodiments, the sleep state, the first wake state, the second wake state, the predefined interval, the monitoring period, the monitoring rate, or any combination thereof is configured to enable monitoring of the subject for at least about 72 hours continuously.

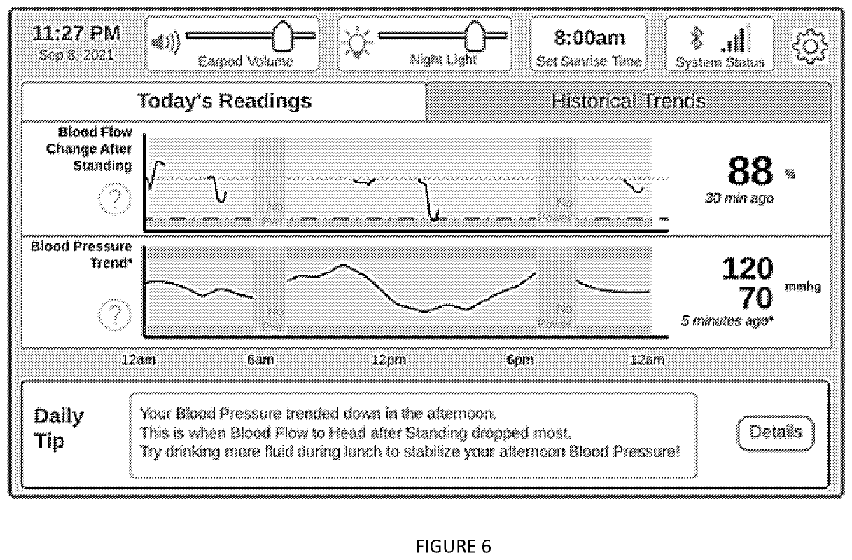

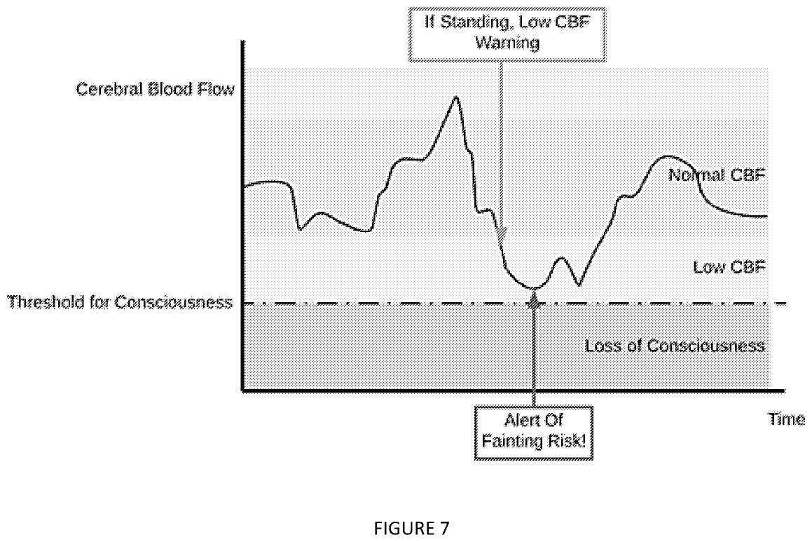

In some embodiments, the local base station 210 further comprises a wireless power transmitter (WPT). In some embodiments, the WPT comprises infrared light emitters. In some embodiments, the WPT is configured to transmit energy 201 between the in-ear device 100 and the base station 210. In some embodiments, the infrared light emitters comprise infrared light-emitting diodes (LEDs). In some embodiments, the local base station 210 does not comprise the wireless power transmitter (WPT). In some embodiments, the local base station 210 further comprises one or more processors configured to transmit an alert via one or more of: SMS, MMS, email, telephone, voice mail, and social media. In some embodiments, the local base station 210 further comprises an acoustic transducer for broadcasting audio messages. In some embodiments, per FIG. 6, the local base station 210 further comprises a screen for displaying biometric information and notifications. In some embodiments, per FIG. 7, the local base station 210 further provides an alert. In some embodiments, the network interface utilizes one or more of: Wi-Fi, 4G, LTE, and 5G. In some embodiments, the computer network comprises the internet.

### Platforms for Long-Term Continuous Monitoring of a Subject

Provided herein is a platform for long-term, continuous monitoring of a subject. In some embodiments, the platform comprises the in-ear device as described in any one or more embodiments herein, the local base station as described in any one or more embodiments herein, and a cloud computing back-end. In some embodiments, the platform is configured for long-term, continuous monitoring of orthostatic hypotension in the subject. In some embodiments, the platform is configured for long-term, continuous monitoring of cerebral blood flow in the subject.

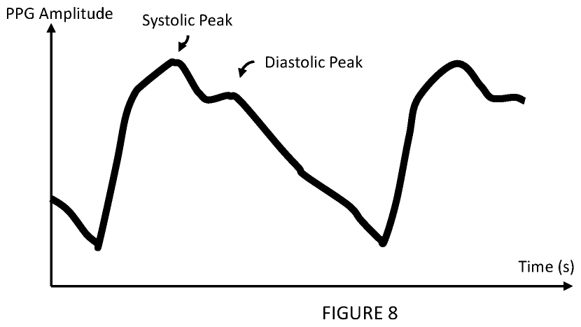

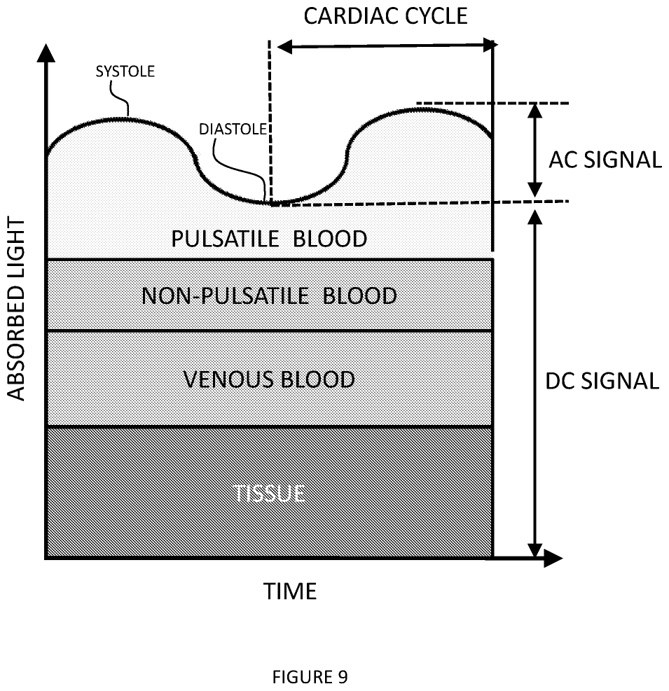

In some embodiments, the network interface is configured to provide connectivity to the cloud computing back-end; and a cloud computing back-end comprising a module configured to store and analyze the biometric and activity data of the subject to identify physiological trends and provide biometric feedback or behavioral coaching recommendations. In some embodiments, the network interface utilizes one or more of: Wi-Fi, 4G, LTE, and 5G. In some embodiments, the computer network comprises the internet. In some embodiments, per FIGS. 7-9, the physiological trends comprise intraday and interday cerebral blood flow trends. In some embodiments, the behavioral coaching recommendations pertain to prevention of insufficient cerebral blood flow that may result in a fall. In some embodiments, the platform is configured for long-term, continuous monitoring of cerebral blood flow in the subject. In some embodiments, the physiological trends comprise intraday and interday blood pressure trends. In some embodiments, the behavioral coaching recommendations pertain to keeping the blood pressure within a safe range. In some embodiments, the physiological trends comprise intraday and interday blood volume trends. In some embodiments, the behavioral coaching recommendations pertain to keeping blood volume within a safe range. In some embodiments, the platform is configured for long-term continuous monitoring as a consumer wellness/fitness wearable, providing biometric feedback such as hydration levels and heart rate variability. In some embodiments, the platform is configured to monitor and manage precipitating factors of stroke and dementia, such as unstable cerebral blood flow. In some embodiments, the platform is configured to manage syncope-related hospital emergency room visits through biometric data driven triage. In some embodiments, the platform is configured to manage cerebral oxygenation and hemodynamics in hospital Intensive Care Units (ICU) or Operating Rooms (OR). In some embodiments, the platform is configured as a high adherence, forget-it's-there, remote patient monitor (RPM) for long term care facilities. In some embodiments, the cloud computing back-end further comprises a module configured to provide a healthcare provider portal application allowing access to real time and historical data and trends for one or more subjects. In some embodiments, the cloud computing back-end further comprises a module configured to provide a subject health portal application allowing access to real time and historical data and trends for the subject. In some embodiments, the analysis comprises application of one or more artificial neural networks (ANNs). In some embodiments, the one or more ANNs are configured to predict cerebral blood flow or blood pressure events.

### Methods for Performing Long-Term Continuous Monitoring of a Subject

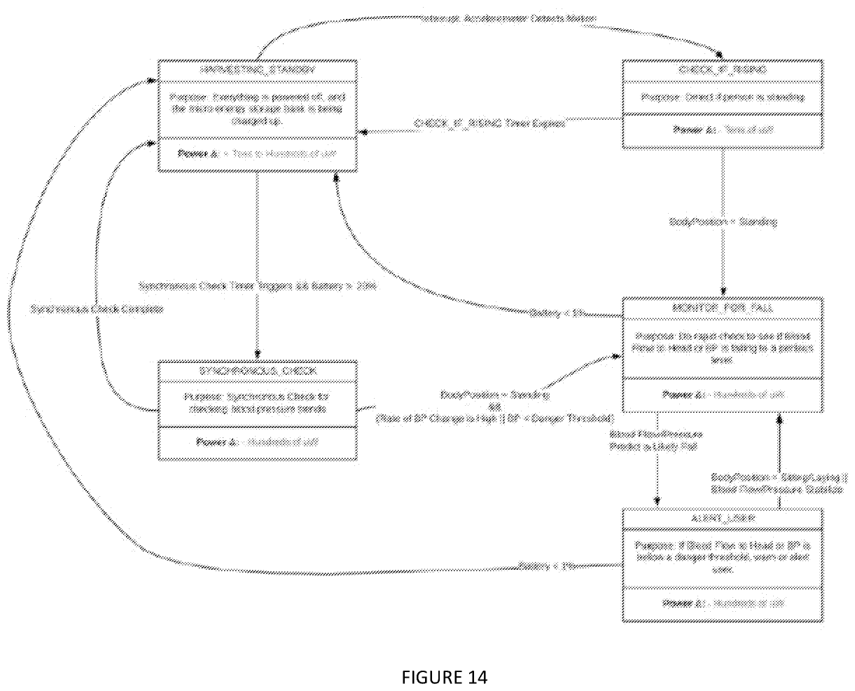

Another aspect provided herein, per FIG. 14, is a method of performing long-term, continuous monitoring of a subject with the in-ear device as provided in any one or more embodiments herein. In some embodiments, the method comprising maintaining the device in a sleep state, shifting the device to a first wake state and shifting the device to a second wake state.

In some embodiments, in the sleep state, the micro energy storage bank is charged by the energy harvesting element. In some embodiments, the device is shifted to the first wake state intermittently. In some embodiments, the device is shifted to the first wake state at a predefined interval. In some embodiments, in the first wake state the micro energy storage bank powers operation of the biometric sensor, the movement sensor, the wireless communications transceiver, or any combination thereof to perform synchronous monitoring of at least one biometric parameter of the subject. In some embodiments, in the first wake state the micro energy storage bank powers operation of the biometric sensor, the movement sensor, the wireless communications transceiver, or any combination thereof to perform synchronous monitoring of at least one activity parameter of the subject. In some embodiments, the predefined interval is between about 1 minute and 30 minutes.

In some embodiments, the device is shifted to the second wake state based on the at least one biometric parameter, the at least one activity parameter, or both. In some embodiments, in the second wake state the micro energy storage bank powers operation of the biometric sensor, the movement sensor, and the wireless communications transceiver. In some embodiments, in the second wake state the micro energy storage bank powers operation of the biometric sensor, the movement sensor, and the wireless communications transceiver to perform asynchronous monitoring of the at least one biometric parameter of the subject. In some embodiments, in the second wake state the micro energy storage bank powers operation of the biometric sensor, the movement sensor, and the wireless communications transceiver to perform asynchronous monitoring of the at least one activity parameter of the subject of the subject. In some embodiments, in the second wake state the micro energy storage bank powers operation of the biometric sensor, the movement sensor, and the wireless communications transceiver to perform asynchronous monitoring of the at least one biometric parameter of the subject and the at least one activity parameter of the subject of the subject.

In some embodiments, in the first wake state or the second wake state, the power consumption of the device is more than the power output of the energy harvesting element. In some embodiments, in the first wake state or the second wake state, the biometric sensor monitors the at least one biometric parameter of the subject at a rate of between about 1 Hz and 200 Hz. In some embodiments, in the first wake state or the second wake state, the movement sensor monitors the at least one activity parameter of the subject at a rate of between about 1 Hz and 200 Hz.

In some embodiments, the at least one biometric parameter of the subject comprises one or more of: cerebral blood flow, blood pressure, blood volume, heart rate, heart rate variability, and blood oxygenation. In some embodiments, the at least one activity parameter of the subject comprises one or more of: activity level, body posture, and change in body posture.

In some embodiments, the method further comprises returning the device to the sleep state. In some embodiments, the method further comprises returning the device to the sleep state after performing the synchronous or asynchronous monitoring of the subject. In some embodiments, the method further comprises returning the device to the sleep state after performing the synchronous or asynchronous monitoring of the subject for a monitoring period. In some embodiments, the monitoring period is between about 5 seconds and 120 seconds.

In some embodiments, the method further comprises monitoring the charge of the micro energy storage bank. In some embodiments, when the charge is below a predetermined threshold, the device is maintained in the sleep state. In some embodiments, when the charge exceeds the is above a second predetermined threshold the device is allowed to shift to the first wake state or the second wake state.

In some embodiments, the charge management protocol is configured for increased energy harvesting and decreased energy use, to enable monitoring of the subject for at least about 24 hours continuously. In some embodiments, the charge management protocol is configured for increased energy harvesting and decreased energy use, to enable monitoring of the subject for at least about 72 hours continuously. In some embodiments, the state management protocol is configured for increased energy harvesting and decreased energy use, to enable monitoring of the subject for at least about 24 hours continuously. In some embodiments, the state management protocol is configured for increased energy harvesting and decreased energy use, to enable monitoring of the subject for at least about 72 hours continuously.

In some embodiments, the sleep state, the first wake state, the second wake state, the predefined interval, the monitoring period, the monitoring rate, or any combination thereof is configured to enable monitoring of the subject for at least about 24 hours continuously. In some embodiments, the sleep state, the first wake state, the second wake state, the predefined interval, the monitoring period, the monitoring rate, or any combination thereof is configured to enable monitoring of the subject for at least about 72 hours continuously.

In some embodiments, the method further comprises receiving from the subject answers to a user survey. As shown, in some embodiments, the user survey receives an indent data, a technical data, a raw sensor data, a user activity data, a health condition data, a health classification data, or any combination thereof.

### Machine Learning

In some embodiments, machine learning algorithms are utilized to process the biometric data and the activity data. In some embodiments, the machine learning algorithm is used to analyze the data to detect or predict one or more of: poor cerebral blood flow, poor blood pressure, presyncope, syncope, and a fall event. In some embodiments, the machine learning algorithm is used to identify a detected or predicted event. In some embodiments, the machine learning algorithm is used to determine a posture of the subject

In some embodiments, the machine learning algorithms utilized herein employ one or more forms of labels including but not limited to human annotated labels and semi-supervised labels. The human annotated labels can be provided by a hand-crafted heuristic. For example, the hand-crafted heuristic can comprise comparing a current blood pressure to a predetermined blood pressure graph. The semi-supervised labels can be determined using a clustering technique to determine poor cerebral blood flow, poor blood pressure, presyncope, syncope, or a fall event similar to those flagged by previous human annotated labels and previous semi-supervised labels. The semi-supervised labels can employ a XGBoost, a neural network, or both.

In some embodiments, the methods and systems herein employ a distant supervision method. The distant supervision method can create a large training set seeded by a small hand-annotated training set. The distant supervision method can comprise positive-unlabeled learning with the training set as the ‘positive’ class. The distant supervision method can employ a logistic regression model, a recurrent neural network, or both. The recurrent neural network can be advantageous for Natural Language Processing (NLP) machine learning.

Examples of machine learning algorithms can include a support vector machine (SVM), a naïve Bayes classification, a random forest, a neural network, deep learning, or other supervised learning algorithm or unsupervised learning algorithm for classification and regression. The machine learning algorithms can be trained using one or more training datasets.

In some embodiments, the machine learning algorithm utilizes regression modeling, wherein relationships between predictor variables and dependent variables are determined and weighted. In one embodiment, for example, a predicted event can be a dependent variable and is derived from the biometric and activity data.

In some embodiments, a machine learning algorithm is used to select catalogue data and recommend project scope. A non-limiting example of a multi-variate linear regression model algorithm is seen below: probability=A₀+A₁(X₁)+A₂(X₂)+A₃(X₃)+A₄(X₄)+A₅(X₅)+A₆(X₆)+A₇(X₇) . . . wherein Aᵢ (A₁, A₂, A₃, A₄, A₅, A₆, A₇, . . . ) are “weights” or coefficients found during the regression modeling; and Xᵢ (X₁, X₂, X₃, X₄, X₅, X₆, X₇, . . . ) are data collected from the User. Any number of Aᵢ and Xᵢ variable can be included in the model. For example, in a non-limiting example wherein there are 3 Xᵢ terms, X₁ is the biometric data, X₂ is the activity data, and X₃ is the probability that an event has been detected or predicted. In some embodiments, the programming language “R” is used to run the model.

In some embodiments, training comprises multiple steps. In a first step, an initial model is constructed by assigning probability weights to predictor variables. In a second step, the initial model is used to “recommend” potential events. In a third step, the validation module accepts verified data regarding the subject's health conditions and feeds back the verified data to improve calculation. At least one of the first step, the second step, and the third step can repeat one or more times continuously or at set intervals.

### Computing System

Computer system 1000 may include one or more processors 1001, a memory 1003, and a storage 1008 that communicate with each other, and with other components, via a bus 1040. The bus 1040 may also link a display 1032, one or more input devices 1033 (which may, for example, include a keypad, a keyboard, a mouse, a stylus, etc.), one or more output devices 1034, one or more storage devices 1035, and various tangible storage media 1036. All of these elements may interface directly or via one or more interfaces or adaptors to the bus 1040. For instance, the various tangible storage media 1036 can interface with the bus 1040 via storage medium interface 1026. Computer system 1000 may have any suitable physical form, including but not limited to one or more integrated circuits (ICs), printed circuit boards (PCBs), mobile handheld devices (such as mobile telephones or PDAs), laptop or notebook computers, distributed computer systems, computing grids, or servers.

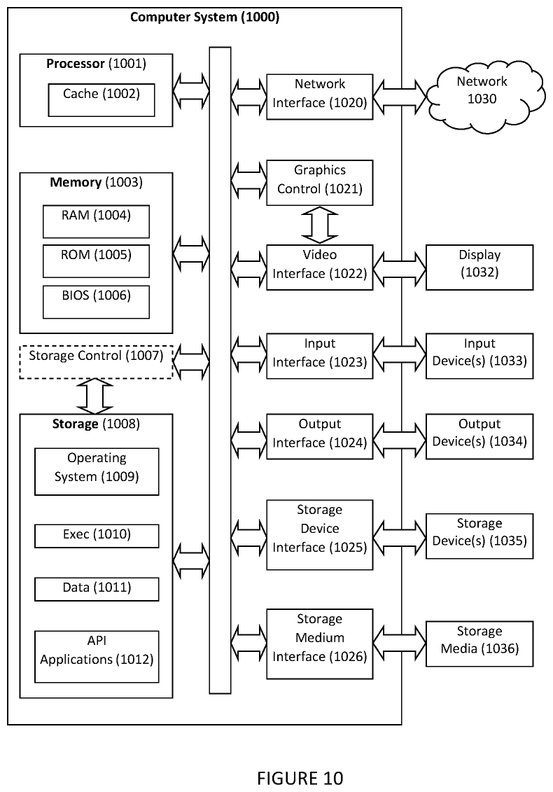

Computer system 1000 includes one or more processor(s) 1001 (e.g., central processing units (CPUs) or general purpose graphics processing units (GPGPUs)) that carry out functions. Processor(s) 1001 optionally contains a cache memory unit 1002 for temporary local storage of instructions, data, or computer addresses. Processor(s) 1001 are configured to assist in execution of computer readable instructions. Computer system 1000 may provide functionality for the components depicted in FIG. 10 as a result of the processor(s) 1001 executing non-transitory, processor-executable instructions embodied in one or more tangible computer-readable storage media, such as memory 1003, storage 1008, storage devices 1035, and/or storage medium 1036. The computer-readable media may store software that implements particular embodiments, and processor(s) 1001 may execute the software. Memory 1003 may read the software from one or more other computer-readable media (such as mass storage device(s) 1035, 1036) or from one or more other sources through a suitable interface, such as network interface 1020. The software may cause processor(s) 1001 to carry out one or more processes or one or more steps of one or more processes described or illustrated herein. Carrying out such processes or steps may include defining data structures stored in memory 1003 and modifying the data structures as directed by the software.

The memory 1003 may include various components (e.g., machine readable media) including, but not limited to, a random access memory component (e.g., RAM 1004) (e.g., static RAM (SRAM), dynamic RAM (DRAM), ferroelectric random access memory (FRAM), phase-change random access memory (PRAM), etc.), a read-only memory component (e.g., ROM 1005), and any combinations thereof. ROM 1005 may act to communicate data and instructions unidirectionally to processor(s) 1001, and RAM 1004 may act to communicate data and instructions bidirectionally with processor(s) 1001. ROM 1005 and RAM 1004 may include any suitable tangible computer-readable media described below. In one example, a basic input/output system 1006 (BIOS), including basic routines that help to transfer information between elements within computer system 1000, such as during start-up, may be stored in the memory 1003.

Fixed storage 1008 is connected bidirectionally to processor(s) 1001, optionally through storage control unit 1007. Fixed storage 1008 provides additional data storage capacity and may also include any suitable tangible computer-readable media described herein. Storage 1008 may be used to store operating system 1009, executable(s) 1010, data 1011, applications 1012 (application programs), and the like. Storage 1008 can also include an optical disk drive, a solid-state memory device (e.g., flash-based systems), or a combination of any of the above. Information in storage 1008 may, in appropriate cases, be incorporated as virtual memory in memory 1003.

In one example, storage device(s) 1035 may be removably interfaced with computer system 1000 (e.g., via an external port connector (not shown)) via a storage device interface 1025. Particularly, storage device(s) 1035 and an associated machine-readable medium may provide non-volatile and/or volatile storage of machine-readable instructions, data structures, program modules, and/or other data for the computer system 1000. In one example, software may reside, completely or partially, within a machine-readable medium on storage device(s) 1035. In another example, software may reside, completely or partially, within processor(s) 1001. In some embodiments, the non-volatile memory stores an age, gender, height, weight, existing diagnosis, previous event, medication, or any combination thereof.

Bus 1040 connects a wide variety of subsystems. Herein, reference to a bus may encompass one or more digital signal lines serving a common function, where appropriate. Bus 1040 may be any of several types of bus structures including, but not limited to, a memory bus, a memory controller, a peripheral bus, a local bus, and any combinations thereof, using any of a variety of bus architectures. As an example and not by way of limitation, such architectures include an Industry Standard Architecture (ISA) bus, an Enhanced ISA (EISA) bus, a Micro Channel Architecture (MCA) bus, a Video Electronics Standards Association local bus (VLB), a Peripheral Component Interconnect (PCI) bus, a PCI-Express (PCI-X) bus, an Accelerated Graphics Port (AGP) bus, HyperTransport (HTX) bus, serial advanced technology attachment (SATA) bus, and any combinations thereof.

In particular embodiments, when computer system 1000 is connected to network 1030, computer system 1000 may communicate with other devices, specifically mobile devices and enterprise systems, distributed computing systems, cloud storage systems, cloud computing systems, and the like, connected to network 1030. Communications to and from computer system 1000 may be sent through network interface 1020. For example, network interface 1020 may receive incoming communications (such as requests or responses from other devices) in the form of one or more packets (such as Internet Protocol (IP) packets) from network 1030, and computer system 1000 may store the incoming communications in memory 1003 for processing. Computer system 1000 may similarly store outgoing communications (such as requests or responses to other devices) in the form of one or more packets in memory 1003 and communicated to network 1030 from network interface 1020. Processor(s) 1001 may access these communication packets stored in memory 1003 for processing.

Examples of the network interface 1020 include, but are not limited to, a network interface card, a modem, and any combination thereof. Examples of a network 1030 or network segment 1030 include, but are not limited to, a distributed computing system, a cloud computing system, a wide area network (WAN) (e.g., the Internet, an enterprise network), a local area network (LAN) (e.g., a network associated with an office, a building, a campus or other relatively small geographic space), a telephone network, a direct connection between two computing devices, a peer-to-peer network, and any combinations thereof. A network, such as network 1030, may employ a wired and/or a wireless mode of communication. In general, any network topology may be used.

In addition or as an alternative, computer system 1000 may provide functionality as a result of logic hardwired or otherwise embodied in a circuit, which may operate in place of or together with software to execute one or more processes or one or more steps of one or more processes described or illustrated herein. Reference to software in this disclosure may encompass logic, and reference to logic may encompass software. Moreover, reference to a computer-readable medium may encompass a circuit (such as an IC) storing software for execution, a circuit embodying logic for execution, or both, where appropriate. The present disclosure encompasses any suitable combination of hardware, software, or both.

Those of skill in the art will appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality.

The various illustrative logical blocks, modules, and circuits described in connection with the embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof configured to perform the functions described herein. A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein may be embodied directly in hardware, in a software module executed by one or more processor(s), or in a combination of the two. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

In accordance with the description herein, suitable computing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will also recognize that select televisions, video players, and digital music players with optional computer network connectivity are suitable for use in the system described herein. Suitable tablet computers, in various embodiments, include those with booklet, slate, and convertible configurations, known to those of skill in the art.

In some embodiments, the computing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smartphone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Those of skill in the art will also recognize that suitable media streaming device operating systems include, by way of non-limiting examples, Apple TV®, Roku®, Boxee®, Google TV®, Google Chromecast®, Amazon Fire®, and Samsung® HomeSync®. Those of skill in the art will also recognize that suitable video game console operating systems include, by way of non-limiting examples, Sony® PS3 ®, Sony® PS4®, Microsoft® Xbox 360®, Microsoft Xbox One, Nintendo® Wii®, Nintendo® Wii U®, and Ouya®.

### Non-Transitory Computer Readable Storage Medium

In some embodiments, the platforms, systems, media, and methods disclosed herein include one or more non-transitory computer readable storage media encoded with a program including instructions executable by the operating system of an optionally networked computing device. In further embodiments, a computer readable storage medium is a tangible component of a computing device. In still further embodiments, a computer readable storage medium is optionally removable from a computing device. In some embodiments, a computer readable storage medium includes, by way of non-limiting examples, CD-ROMs, DVDs, flash memory devices, solid state memory, magnetic disk drives, magnetic tape drives, optical disk drives, distributed computing systems including cloud computing systems and services, and the like. In some cases, the program and instructions are permanently, substantially permanently, semi-permanently, or non-transitorily encoded on the media.

### Computer Program

In some embodiments, the platforms, systems, media, and methods disclosed herein include at least one computer program, or use of the same. A computer program includes a sequence of instructions, executable by one or more processor(s) of the computing device's CPU, written to perform a specified task. Computer readable instructions may be implemented as program modules, such as functions, objects, Application Programming Interfaces (APIs), computing data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft® .NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, my SQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

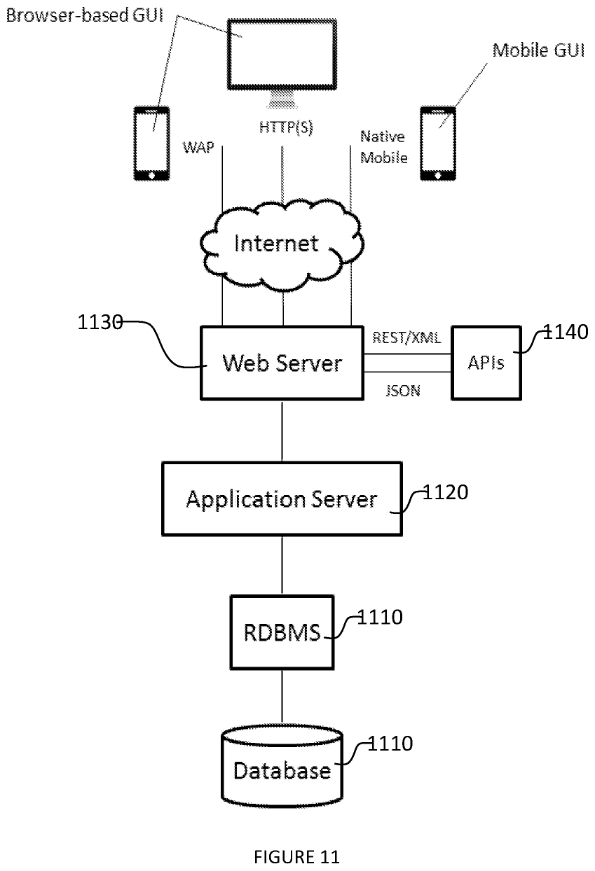

Referring to FIG. 11, in a particular embodiment, an application provision system comprises one or more databases 1100 accessed by a relational database management system (RDBMS) 1110. Suitable RDBMSs include Firebird, MySQL, PostgreSQL, SQLite, Oracle Database, Microsoft SQL Server, IBM DB2, IBM Informix, SAP Sybase, SAP Sybase, Teradata, and the like. In this embodiment, the application provision system further comprises one or more application severs 1120 (such as Java servers, .NET servers, PHP servers, and the like) and one or more web servers 1130 (such as Apache, IIS, GWS and the like). The web server(s) optionally expose one or more web services via app application programming interfaces (APIs) 1140. Via a network, such as the Internet, the system provides browser-based and/or mobile native user interfaces.

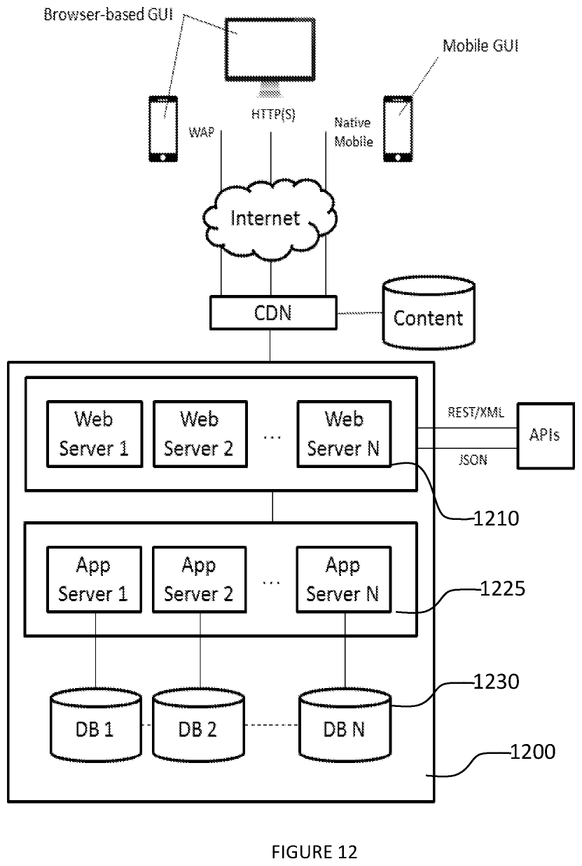

Referring to FIG. 12, in a particular embodiment, an application provision system alternatively has a distributed, cloud-based architecture 1200 and comprises elastically load balanced, auto-scaling web server resources 1210 and application server resources 1220 as well synchronously replicated databases 1230.

### Mobile Application

In some embodiments, a computer program includes a mobile application provided to a mobile computing device. In some embodiments, the mobile application is provided to a mobile computing device at the time it is manufactured. In other embodiments, the mobile application is provided to a mobile computing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C #, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, Airplay SDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Google® Play, Chrome Web Store, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable complied applications.

### Web Browser Plug-In

In some embodiments, the computer program includes a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. In some embodiments, the toolbar comprises one or more web browser extensions, add-ins, or add-ons. In some embodiments, the toolbar comprises one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™ PHP, Python™, and VB .NET, or combinations thereof.

Web browsers (also called Internet browsers) are software applications, designed for use with network-connected computing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called microbrowsers, mini-browsers, and wireless browsers) are designed for use on mobile computing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® Web OS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

### Software Modules

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on a distributed computing platform such as a cloud computing platform. In some embodiments, software modules are hosted on one or more machines in one location. In other embodiments, software modules are hosted on one or more machines in more than one location.

### Databases

In some embodiments, the platforms, systems, media, and methods disclosed herein include one or more databases, or use of the same. In view of the disclosure provided herein, those of skill in the art will recognize that many databases are suitable for storage and retrieval of medical information. In various embodiments, suitable databases include, by way of non-limiting examples, relational databases, non-relational databases, object oriented databases, object databases, entity-relationship model databases, associative databases, and XML databases. Further non-limiting examples include SQL, PostgreSQL, MySQL, Oracle, DB2, and Sybase. In some embodiments, a database is internet-based. In further embodiments, a database is web-based. In still further embodiments, a database is cloud computing-based. In a particular embodiment, a database is a distributed database. In other embodiments, a database is based on one or more local computer storage devices.

### Terms and Definitions

Unless otherwise defined, all technical terms used herein have the same meaning as commonly understood by one of ordinary skill in the art to which this disclosure belongs.

As used herein, the singular forms “a,” “an,” and “the” include plural references unless the context clearly dictates otherwise. Any reference to “or” herein is intended to encompass “and/or” unless otherwise stated.

As used herein, the term “in-ear” in some cases refers to being on or attached to the ear of a subject. As used herein, the term “in-ear” in some cases refers to being inside the concha of the ear of a subject. As used herein, the term “in-ear” in some cases refers to being inside an ear canal of the subject.

As used herein, the term “about” in some cases refers to an amount that is approximately the stated amount.

As used herein, the term “about” refers to an amount that is near the stated amount by 10%, 5%, or 1%, including increments therein.

As used herein, the term “about” in reference to a percentage refers to an amount that is greater or less the stated percentage by 10%, 5%, or 1%, including increments therein.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

### EXAMPLES

The following illustrative examples are representative of embodiments of the software applications, systems, and methods described herein and are not meant to be limiting in any way.

### Example 1

Grandma Sarah has orthostatic hypotension and often falls after standing up due to a loss of blood flow to her head. Sarah puts in an in-ear device. This device is intended for long-term, continuous use where Sarah can have the device sit in her cymba concha for at least up to a week without removing it. The device remains in Sarah's ear as Sarah sleeps, eats, bathes, and performs her day-to-day activities. Because the device is so small and Sarah does not have to do anything to upkeep the device, Sarah forgets it is there. Sarah does not even have to remember to recharge the device because the device is continuously energy harvesting while in her ear. The device is able to function for long periods of time despite its small size because the device is continuously energy harvesting while being worn, and because, the device switches back and forth between different sleep and wake states. The first wake state occurs at predefined intervals—once every 3 minutes—whereby the device powers up from a sleeping state to take note of Sarah's biometrics. In addition to the first wake state, the second wake state powers up whenever Sarah moves enough to suggest she is about to stand up. In the second wake state, the device monitors whether Sarah is indeed transitioning to a standing position. It turns out that Sarah was just repositioning herself while she was lounging on the couch and does not transition into a standing position, so the device goes back to sleep to conserve energy and harvest energy efficiently. Later in the day, Sarah gets up to walk to the kitchen to prepare lunch, at which point the device again wakes up to the second wake state, notices her body position has changed into a standing state, then rapidly monitors to understand how blood flow and blood pressure to her head is changing as she stands up. The energy harvesting element of Sarah's device ensures that the in-ear device always has enough charge to measure how her body responds in the minutes following a transition to standing event.

In some embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure.

1. An in-ear device for long-term, continuous monitoring of a subject, the device comprising:
a) a biometric sensor configured to monitor at least one biometric parameter of the subject;
b) a movement sensor configured to monitor at least one activity parameter of the subject;
c) an energy storage bank;
d) a wireless communications transceiver; and
e) an attachment mechanism for securing the device to an auricle of the subject;
wherein the biometric sensor, the movement sensor, the energy storage bank, and the wireless communications transceiver are adapted to fit within a volume 20 mm or less in its longest dimension and attach to the auricle of the subject at a cymba concha of the subject.

2. (canceled)

3. The in-ear device of claim 1, configured to monitor the subject for at least about 24 hours continuously or for at least about 72 hours continuously.

4. (canceled)

5. The in-ear device of claim 1, wherein the energy storage bank comprises a supercapacitor or a micro battery.

6. (canceled)

7. The in-ear device of claim 1, further comprising an energy harvesting element configured to charge the energy storage bank, and wherein the energy harvesting element compromises one or more of:
a) a photovoltaic cell configured to harvest energy from natural daylight, interior lighting, infrared emitters, or a combination thereof;
b) a RF antenna configured to harvest energy from the environment of the device;
c) a thermoelectric generator configured to harvest energy from body heat of the subject and
d) a piezoelectric material configured to harvest energy from motion of the subject.

8. (canceled)

9. (canceled)

10. (canceled)

11. The in-ear device of claim 1, further comprising a logic element performing a charge management protocol comprising:
a) monitoring the charge of the micro energy storage bank;
b) when the charge is below a predetermined threshold, allowing the energy harvesting element to charge the energy storage bank; and
c) when the charge is above a second predetermined threshold, allowing the energy storage bank to power operation of the biometric sensor, the movement sensor, or the wireless communications transceiver.

12. The in-ear device of claim 1, further comprising a logic element performing a state management protocol comprising:
a) maintaining the device in a sleep state, wherein the energy storage bank is charged;
b) shifting the device to a first wake state intermittently, at a predefined interval, wherein the energy storage bank powers operation of the biometric sensor, the movement sensor, and the wireless communications transceiver to perform synchronous monitoring of the subject; and
c) shifting the device to a second wake state as a response to the at least one biometric parameter, the at least one activity parameter, or both, wherein the energy storage bank powers operation of the biometric sensor, the movement sensor, and the wireless communications transceiver to perform asynchronous monitoring of the subject.

13. (canceled)

14. (canceled)

15. (canceled)

16. The in-ear device of claim 12, further comprising an energy harvesting element configured to charge the energy storage bank, and wherein, in the first wake state or the second wake state, the power consumption of the device is more than the power output of the energy harvesting element.

17. (canceled)

18. (canceled)

19. The in-ear device of claim 1, wherein the attachment mechanism is a biocompatible adhesive.

20. The in-ear device of claim 1, wherein the attachment mechanism is one or more elastomeric wings with a durometer less than 80 Shore A, configured to extend into the triangular fossa while pressing into the helix, and/or to extend into the concha cavum while pressing into the antihelix or antitragus.

21. (canceled)

22. The in-ear device of claim 1, wherein the attachment mechanism is one or more elastomeric rough surface finishes with a durometer less than 80 Shore A, configured to maximize the presence of Van Der Waals forces.

23. (canceled)

24. The in-ear device of claim 1, wherein the attachment mechanism is a set of elastomeric appendages with a durometer less than 50 Shore A, configured to maximize friction by press into the helix, antihelix, antitragus, and/or the cymba concha of the subject.

25. The in-ear device of claim 1, wherein the attachment mechanism is a custom molded elastomer configured to lock into the subject's unique concha morphology.

26. (canceled)

27. (canceled)

28. (canceled)

29. (canceled)

30. (canceled)

31. The in-ear device of claim 1, wherein the biometric sensor comprises an optical sensor.

32. The in-ear device of claim 31, wherein the optical sensor comprises a photoplethysmography (PPG) sensor.

33. The in-ear device of claim 1, wherein the movement sensor comprises at least one accelerometer.

34. The in-ear device of claim 1, wherein the movement sensor comprises at least one altimeter.

35. The in-ear device of claim 1, wherein the at least one biometric parameter of the subject comprises one or more of: cerebral blood flow, blood pressure, blood volume, heart rate, heart rate variability, and blood oxygenation.

36. The in-ear device of claim 1, wherein the at least one activity parameter of the subject comprises one or more of: motion, activity level, body posture, and change in body posture.

37. The in-ear device of claim 1, wherein the wireless communications transceiver utilizes a Near-Field Communication (NFC) protocol, Bluetooth, Bluetooth Low Energy, LoRa, or Wi-Fi.

38. (canceled)

39. (canceled)

40. The in-ear device of claim 1, wherein the device further comprises an acoustic transducer for communicating audio messages with low sound leakage perceived by others near the subject.

41. (canceled)

42. (canceled)

43. The in-ear device of claim 1, further comprising a microcontroller configured to analyze the at least one biometric parameter and the at least one activity parameter.

44. The in-ear device of claim 43, wherein the analysis comprises application of one or more artificial neural networks (ANNs).

45. The in-ear device of claim 44, wherein the one or more ANNs are configured to detect or predict one or more of: poor cerebral blood flow, poor blood pressure, presyncope, syncope, and a fall event.

46. The in-ear device of claim 44, wherein the one or more ANNs are configured to infer systolic and diastolic blood pressure from the at least one biometric sensor or activity sensor.

47. The in-ear device of claim 43, wherein the analysis comprises identification of physiological trends.

48. The in-ear device of claim 47, wherein the physiological trends comprise trends in one or more of: cerebral blood flow, blood pressure, blood volume, heart rate, heart rate variability, blood oxygenation, and activity level.

49. The in-ear device of claim 47, wherein the physiological trends are interday and intraday trends.

50.-86. (canceled)
